(19) United States
(12) Patent Application Publication
(10) Pub. No.: US 2022/0218941 A1
(43) Pub. Date: Jul. 14, 2022

(54) A Wearable System for Behind-The-Ear Sensing and Stimulation

(71) Applicant: The Regents of The University of Colorado, a Body Corporate, Denver, CO (US)

(72) Inventors: Tam Vu, Boulder, CO (US); Robin Deterding, Boulder, CO (US); Ann Halbower, Salida, CO (US); Nhat Pham, Boulder, CO (US); Nam Ngoc Bui, Broomfield, CO (US); Farnoush Banaei-Kashani, Centennial, CO (US)

(21) Appl. No.: 17/610,419

(22) PCT Filed: May 6, 2020
(86) PCT No.: PCT/US20/31712

§ 371 Date: Nov. 10, 2021

Related U.S. Application Data
(60) Provisional application No. 62/844,432, filed on May 7, 2019.
(60) Provisional application No. 62/900,183, filed on Sep. 13, 2019.
(60) Provisional application No. 62/988,336, filed on Mar. 11, 2020.

(51) Int. Cl. A61M 21/00

(52) U.S. Cl. CPC A61M 21/00; A61M 2021/0027; A61M 2021/0055; A61M 2021/0072; A61M 2021/0083; A61M 2021/0044

(57) ABSTRACT

Various embodiments provide novel tools and techniques for behind-the-car biosignal sensing and stimulation. A system includes a behind-the-car wearable device further including an ear piece, one or more sensors coupled to a patient behind the ears of the patient and a host machine coupled to the behind-the-car wearable device. The host machine includes a second processor, and a second computer readable medium in communication with the second processor, the second computer readable medium having encoded thereon a second set of instructions executable by the second processor to obtain the first signal, separate the first signal into one or more individual biosignals, and determine, based on one or more features extracted from the one or more individual biosignals, awakefulness classification of the patient.

### CROSS REFERENCE TO RELATED APPLICATIONS

This application claims priority to U.S. Provisional patent application Ser No. 62/844,432, filed May 7, 2019 by Tam Vu et al. (attorney docket no. 1171.05PR), entitled “System and Apparatus for a Non-Invasive Multimodal and Continuous Biosignal Measurement and Stimulation Device,” U.S. Provisional Patent Application Ser. No. 62/900,183, filed Sep. 13, 2019 by Tam Vu et al. (attorney docket no. 1171.05PR2), entitled “A Wearable System for Behind-the-Ear Sensing and Stimulation,” and U.S. Provisional Patent Application Ser. No. 62/988,336, filed Mar. 11, 2020 by Tam Vu et al (attorney docket no 1171.05PR3), entitled “A Wearable System for Behind-The-Ear Sensing and Stimulation,” the entire disclosures of which are incorporated herein by reference in their entirety for all purposes.

### BACKGROUND

Computers and computing systems have affected nearly every aspect of modern living. Computers are generally involved in work, recreation, healthcare, transportation, entertainment, household management, etc. In recent years, wearable computing devices have experienced explosive market growth. This growth has been driven, at least in part, by the miniaturization of computing components and sensors. Many conventional wearables integrate sensor technology to provide health information such as heart rate. This information can be continuously gathered by the wearable as the user goes through his or her normal daily activities. The ability to gather additional biosignal information and process the gathered information presents several significant challenges in the art.

Excessive daytime sleepiness (EDS) affects over half of the world's population. EDS often results in frequent lapses in awareness of the environment. Typically, a sleep study or other form of patient monitoring is utilized to diagnose and treat EDS. Polysomnography (PSG) and camera-based solutions, for example, have been used to detect EDS. In particular, the Maintenance of Wakefulness Test (MWT), which uses PSG, sets the standard for quantifying microsleep based on electrical signals from the human head, such as brain waves, eye ball movements, chin muscle tone, and behaviors such as eyelid closures, eye blinks, and head nods. Conventionally, a complicated setup of equipment and sensors is required to administer the MWT. Furthermore, the MWT must be conducted by technicians in a clinical environment. Camera-based solutions for detecting microsleep only capture eyelid closures, head nods, and externally manifested physiological signs of sleepiness.

Thus, novel tools and techniques for behind-the-ear sensing and stimulation for EDS monitoring are provided. The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

At least one embodiment comprises a behind-the-ear sensor. The behind-the-ear sensor comprises a shape and size that is configured to rest behind a user's ear. The behind-the-ear sensor further comprising one or more of the following sensor components: an inertial measurement unit, an LED and photodiode, a microphone, or a radio antenna. The earbud sensor is configured to non-invasively measure various biosignals such as brain waves (electroencephalogram (EEG) and electromagnetic fields generated by neural activities), eyes movements (electrooculography (EOG)), facial muscle activities (electromyography (EMG)), electrodermal activity (EDA), head motion, heart rate, breathing rate, swallowing sound, voice, and organ sounds from behind the user's ear.

Additionally, at least one embodiment comprises a computer system for separating signals from a biosignal received from a behind-the-ear sensor based on transfer learning and deep neural networks. The separated signals comprise one or more of EEG, EOG and EMG signals from the mixed-signal. The deep neural network is trained without the need of per-user training and is trained on general data from a large population to learn common patterns among people.

Further, at least one embodiment comprises a computer system for quantify, using a machine learning model, human wakefulness levels based on the captured biosignals from behind-the-ears. The computer system can provide stimulation feedback to a user to warn them when they are drowsy or stimulate their brain to keep them awake. For example, the computer system can do one or more of the following: generate, with an array of electrical electrodes, electrical or magnetic signals; emit, with an RF antenna, electromagnetic radiation to stimulate different parts of the brain; produce, with a bone conductance speaker, acoustic or ultra-sound signals to coach the user, and emit, with a light source, various light frequencies to produce different effects on the brain.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

A further understanding of the nature and advantages of particular embodiments may be realized by reference to the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1A is a schematic block diagram of a behind-the-ear sensing and stimulation system, in accordance with various embodiments.

FIG. 1B is a functional block diagram of a behind-the-ear sensing and stimulation system, in accordance with various embodiments.

FIG. 2 is a schematic perspective view diagram of a behind-the-ear sensing and stimulation system worn by a patient, in accordance with various embodiments.

FIG. 3 is a schematic circuit diagram of three-fold cascaded amplifying active electrode circuit in a behind-the-ear sensing and stimulation system, in accordance with various embodiments.

FIG. 4A is a graph depicting raw and filtered signals gathered by the behind-the-ear sensing and stimulation system after a buffering stage, in accordance with various embodiments.

FIG. 4B is a graph depicting a signal spectrum of the signal produced by the behind-the-ear sensing and stimulation system after a feed forward differential pre-amplifying, in accordance with various embodiments.

FIG. 4C is a graph depicting raw signals with and without adaptive gain control of the adaptive amplifying stage, in accordance with various embodiments.

FIG. 5A is a graph depicting oversampling and averaging of a saccadic eye movement signal, in accordance with various embodiments.

FIG. 5B illustrates comparative graphs of EEG, vertical EOG, and horizontal EOG signals, in accordance with various embodiments.

FIG. 6 is a schematic depiction of a layout of electrodes on a human head, in accordance with various embodiments.

FIG. 7 is a schematic block diagram of a behind-the-ear sensing and stimulation system, in accordance with various embodiments.

FIG. 8 is a schematic representation of a machine learning model for the behind-the-ear sensing and stimulation system, in accordance with various embodiments.

FIG. 9 is a table of features that may be extracted from signals captured by the behind-the-ear sensing and stimulation system, in accordance with various embodiments.

FIG. 10 is a flow diagram of a method for the behind-the-ear sensing and stimulation system, in accordance with various embodiments.

FIG. 11 is a schematic block diagram of a computer system for behind-the-ear sensing and stimulation, in accordance with various embodiments.

FIG. 12 is a schematic block diagram illustrating system of networked computer devices, in accordance with various embodiments.

### DETAILED DESCRIPTION

While various aspects and features of certain embodiments have been summarized above, the following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” in this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

In an aspect, a system for behind-the-ear sensing and stimulation is provided. The system includes a behind-the-ear wearable device and a host machine. The behind-the-ear wearable device may include an ear piece configured to be worn behind an ear of a patient, one or more sensors coupled to the ear piece and configured to be in contact with the skin of the patient behind the ear of the patient, a first processors coupled to the one or more sensors, and a first computer readable medium in communication with the first processor, the first computer readable medium having encoded thereon a first set of instructions executable by the first processor to obtain, via the one or more sensors, a first signal, wherein the first signal comprises one or more combined biosignals. the behind-the-ear wearable device may further be configured to pre-process the first signal and transmit the first signal to the host machine.

The host machine may be coupled to the behind-the-ear wearable device. The host machine may further include a second processor; and a second computer readable medium in communication with the second processor, the second computer readable medium having encoded thereon a second set of instructions executable by the second processor to obtain, via the behind-the-ear wearable device, the first signal. The second processor may further execute the instructions to separate the first signal into one or more individual biosignals, identify, via a machine learning model, one or more features associated with a wakefulness state, and extract the one or more features from each of the one or more individual biosignals. Based on the one or more features extracted from the one or more individual biosignals, a wakefulness classification of the patient may be determined by the host machine.

In another aspect, an apparatus for behind-the-ear sensing and stimulation is provided. The apparatus includes a processor; and a computer readable medium in communication with the processor, the computer readable medium having encoded thereon a set of instructions executable by the processor to obtain, via one or more behind-the-ear sensors, a first signal collected from behind the ear of a patient, the first signal comprising one or more combined biosignals, and separate the first signal into one or more individual component biosignals. The instructions may further be executed by the processor to identify, via a machine learning model, one or more features associated with a wakefulness state for each of the one or more individual component biosignals, extract the one or more features from each of the one or more individual biosignals, and determine, based on the one or more features extracted from the one or more individual biosignals, a wakefulness classification of the patient.

In a further aspect, a method for behind-the-ear sensing and stimulation is provided. The method includes obtaining, via one or more behind-the-ear sensors, a first signal from behind the ears of a patient, the first signal comprising one or more combined biosignals, and separating, via a machine learning model, the first signal into one or more individual component biosignals. The method may continue by identifying, via the machine learning model, one or more features associated with a wakefulness state for each of the one or more individual component biosignals, and extracting the one or more features from each of the one or more individual biosignals. the method may further include determining, via the machine learning model, a wakefulness classification of the patient based on the one or more features extracted from the one or more individual biosignals.

Disclosed embodiments include a novel compact, light-weight, unobtrusive and socially acceptable design of an ear-based wearable system, which can non-invasively measure various biosignals. As used herein, “biosignals” comprise any health-related metric that is gathered by electronic sensors, such as brain waves (EEG), eyes movements (EOG), facial muscle activities (EMG), electrodermal activity (EDA), and head motion from the area behind human ears. Other potential biosignals that can be captured from behind the ear may include but not limited to heart rate, respiratory rate, sounds from inner body such as speech, breathing sounds, and organ's sounds. These biosignals are captured by different types of sensors in contact with user from behind the ear. In at least one embodiment the sensors may comprise one or more of the following conductive pieces of material that can conduct electrical signals, a microphone which can be in the form of a bone-conductive microphone, a MEMS microphone, an electret microphone, and/or a light-based sensor including a photodetector and light source.

Additionally, in at least one embodiment, a signal separation algorithm based on various machine learning and artificial intelligent techniques, including but not limited to transfer learning, deep neural network, and explainable learning, are used to separate the mixed biosignal captured from around the ear Additionally, the signal separation algorithm may learn from general ‘ground-truth’ from a previously captured signal from a different device. Thus, the system eliminates the need of per-user training for signal separation Based on the sensing results, the system can provide brain stimulation with acoustic signals, visible or infra-red light, and electrical, magnetic, or electromagnetic waves. These stimulations are generated by the bone-conductance speaker, light emitter, and/or array of electrical electrodes.

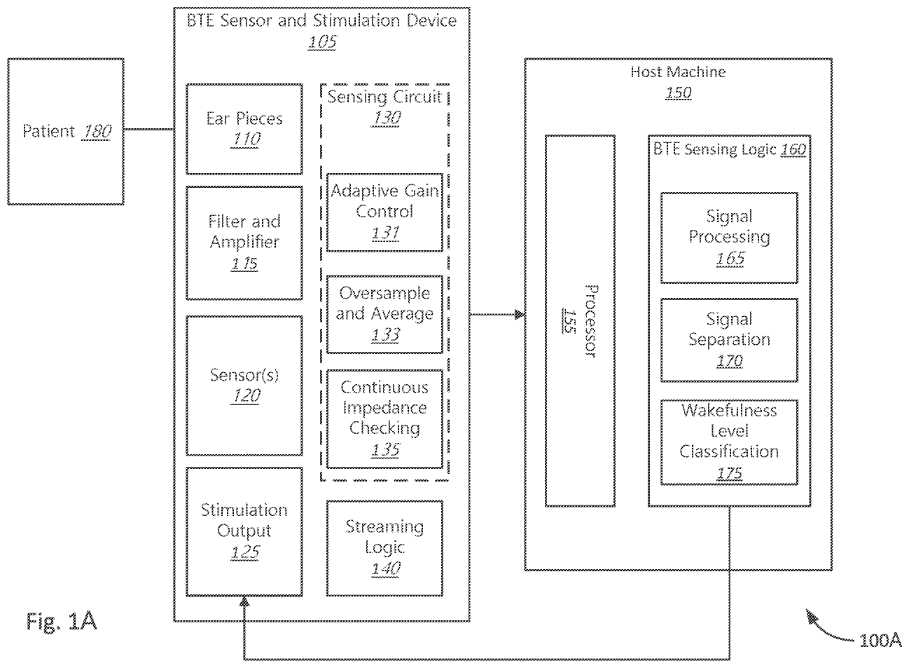

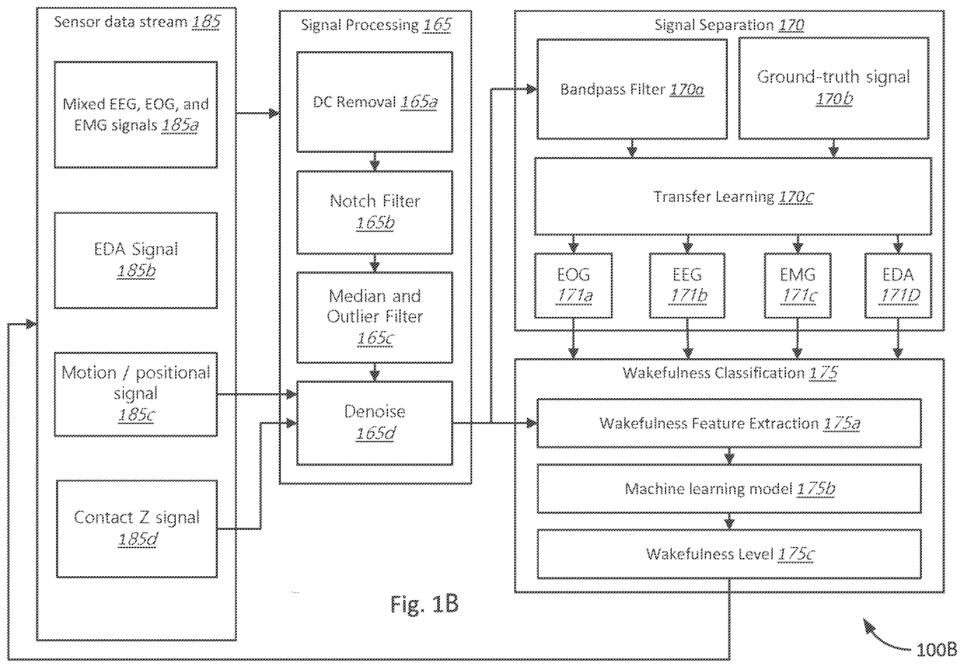

FIG. 1A is a schematic block diagram of a behind-the-ear sensing and stimulation system 100A, in accordance with various embodiments. The system 100A includes a behind-the-ear (BTE) sensor and stimulation device 105 (hereinafter referred to interchangeably as “BTE device 105,” for brevity), silicone ear pieces 110, filter and amplifier 115, one or more sensors 120, stimulation output 125, signal conditioning logic 130, further comprising an adaptive gain control logic 133, oversampling and averaging logic 135, and continuous impedance checking logic 137, streaming logic 140, host machine 150, processor 155, BTE sensing logic 160, signal processing logic 165, signal separation logic 170, wakefulness level classification logic 175, and patient 180. It should be noted that the various components of the system 100A are schematically illustrated in FIG. 1A, and that modifications to the system 100A may be possible in accordance with various embodiments.

In various embodiments, the BTE device 105 may include silicone ear pieces 110, filter and amplifier 115, one or more sensor(s) 120, stimulation output 125, and a sensing circuit 130. The sensing circuit 130 may further include adaptive gain control stage 135, continuous impedance checking 130, and streaming logic 145. The BTE device 105 may be coupled to the patient 180, and host machine 150. Specifically, the silicone ear pieces 110 may be coupled to the patient 180, the silicone ear pieces 110 further including the one or more sensors 120, which may be coupled to the skin of the patient 180 via the silicone ear pieces 110. The host machine 150 may further include a processor 155 and BTE sensing logic 160. BTE sensing logic 160 may include signal processing logic 165, signal separation logic 170, and wakefulness level classification logic 175.

In some embodiments, the BTE device 105 comprises ear-based multimodal sensing hardware and logic that supports sensing quality management and data streaming to control the hardware. BTE sensing logic 160 running on one or more host machines 150 may be configured to process the collected data from the one or more sensors 120 of the BTE device 105. In various embodiments, the BTE device 105 may include one or more silicone ear pieces 110 configured to be worn around respective one or more ears of a patient 180 using the BTE device 105. The one or more silicone ear pieces 110 may each respectively be coupled to on-board circuitry, including, without limitation, a processor such as a microcontroller (MCU), single-board computer, field programmable gate array (FPGA), and custom integrated circuits (IC). The on-board circuitry may further include the filter and amplifier 115, sensing circuit 130, and streaming logic 140.

In various embodiments, the one or more ear pieces 110 may be constructed from a polymeric material. Suitable polymeric materials may include, without limitation, silicone, rubber, polycarbonate (PC), polyvinyl chloride (PVC), thermoplastic polyurethate (TPU), among other suitable polymer materials. In other embodiments, the one or more ear pieces 110 may be constructed from other materials, such as various metals, ceramics, or other composite materials. The one or more ear pieces 110 may be configured to be coupled to a respective ear of a patient 180. For example, in some embodiments, the one or more ear pieces 110 may comprise a hook-like or C-shaped structure, configured to be worn behind-the-ear, around the earlobe. Accordingly, in some embodiments, the one or more ear pieces 110 may be configure to be secured in a hook-like manner around a respective earlobe of the patient 180. In some embodiments, the one or more ear pieces 110 may hang from the earlobes of the patient 180. In some further embodiments, the one or more ear pieces 110 may alternatively comprise a hoop-like structure, configured to be worn around the ear and/or earlobe of the patient 180. In yet further embodiments, the one or more ear pieces 110 may comprise a cup-like structure, configured to be worn over the ear, covering the entirety of the ear and earlobe, making contact with the skin behind the earlobe of the patient 180. In another embodiment, the one or more ear pieces 110 may further comprise an insertion portion configured to be inserted, at least partially, into the ear canal of the patient 180, aiding in securing the one or more ear pieces to the ear of the patient 180.

In some embodiments, the one or more ear pieces 110 may rely on friction between the body of the ear piece 110 and the skin of the patient 180. Thus, friction may stabilize and hold in place the ear piece against the skin of the patient 180, which may be in addition to the attachment to earlobe. In other embodiments, the one or more ear pieces 110 may further include an adhesive material configured to temporarily affix the one or more ear piece 110 against the skin of the patient 180. In yet further embodiments, the one or more ear pieces 110 may include a memory material configured to maintain a physical configuration into which it is manipulated. For example, in some embodiments, the one or more ear pieces 110 may an internal metal wire (e.g., a memory wire), configured to maintain a shape into which each of the one or more ear pieces 110 is manipulated. In yet further embodiments, the one or more ear pieces 110 may further include memory foam material to aid in the one or more ear pieces 110 to conform to the shape of the ear of the patient 180.

In various embodiments, the one or more ear pieces 110 may respectively comprise one or more sensors 120 configured to collect biosignals from the patient 180. In various embodiments, the one or more sensors 120 may be positioned within the ear pieces so as to make contact with the skin of the patient 180. For example, in some embodiments, the one or more sensors 120 may be configured to make contact with the skin of the patient 180 behind the earlobe of the patient 180. In some examples, this may include at least one sensor of the one or more sensors 120 being in contact with the skin over the respective mastoid bones of the patient 180. In various embodiments, the one or more sensors 120 may include various types of sensors, including, without limitation, contact electrodes and other electrode sensors (such as, without limitation, silver fabric, copper pad, or gold-plated copper pad), optical sensors and photodetectors (including a light source for measurement), microphones and other sound detectors (e.g., a bone-conductive microphone, a MEMS microphone, an electret microphone), water sensors, pH sensors, salinity sensors, skin conductance sensors, heart rate monitors, pulse oximeters, and other physiological signal sensors. In yet further embodiments, the one or more sensors 120 may further include one or more positional sensors and/or motion sensors, such as, without limitation, an accelerometer, gyroscope, inertial measurement unit (IMU), global navigation satellite system (GNSS) receiver, or other suitable sensor.

Accordingly, in various embodiments, the one or more sensors 120 may be configured to detect one or more biosignals, including, without limitation, brain waves (EEG), eyes movements (EXG), facial muscle activities (EMG), electrodermal activity (EDA), and head motion from the area behind human ears. Further biosignals may include heart rate, respiratory rate, sounds from inner body such as speech, breathing sounds, and organ's sounds. In some embodiments, the one or more sensors 120 may be configured to capture the various biosignals respectively through different types of sensors. In some embodiments, the one or more sensors 120 may be configured to measure the one or more biosignals from behind-the-ear.

In some embodiments, the one or more sensors 120 may further be incorporated into other structures in addition to, or in place of, the one or more ear pieces 110. For example, in some embodiments, the one or more sensors 120 may further be included in a device, such as a mask, goggles, glasses, cap, hat, visor, helmet, or headband. Thus, the one or more sensors 120 may be configured to be coupled to various parts of the body of the patient 180. As previously described, in various embodiments, the one or more sensors 120 may be configured to be coupled to skin behind-the-ears of the patient 180. In some embodiments, the one or more sensors 120 may further be configured to other parts of the patient, including, without limitation, the eyes, eyelids, and surrounding areas areas around the eyes, forehead, temple, the mouth and areas around the mouth, chin, scalp, and neck of the patient 180. As with the one or more ear pieces 110, in some embodiments, the one or more sensors 120 may further include adhesive material to attach the one or more sensors 120 to the skin of the patient.

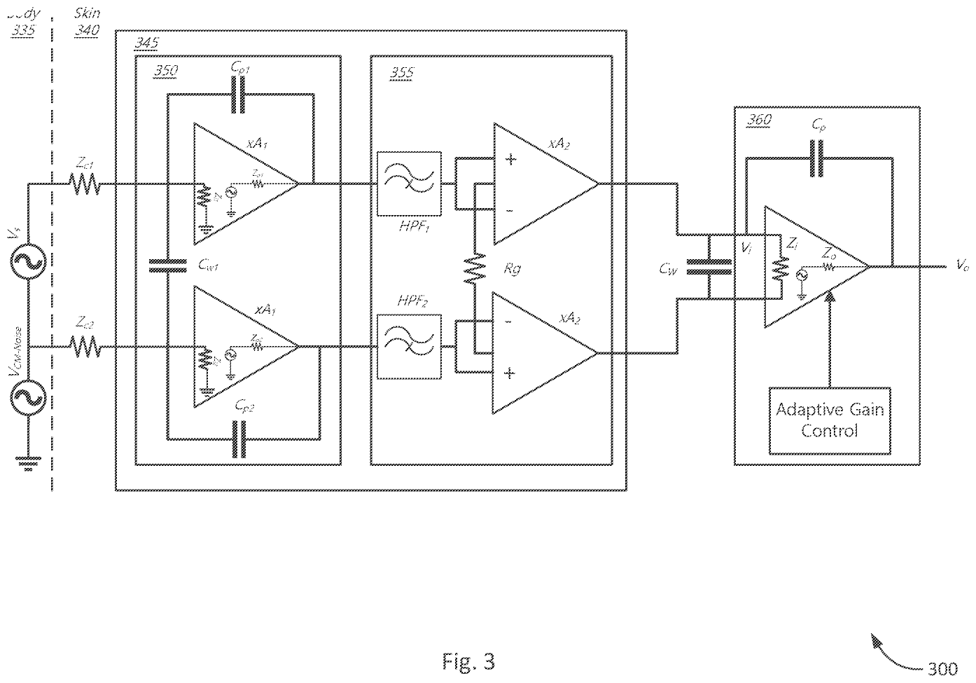

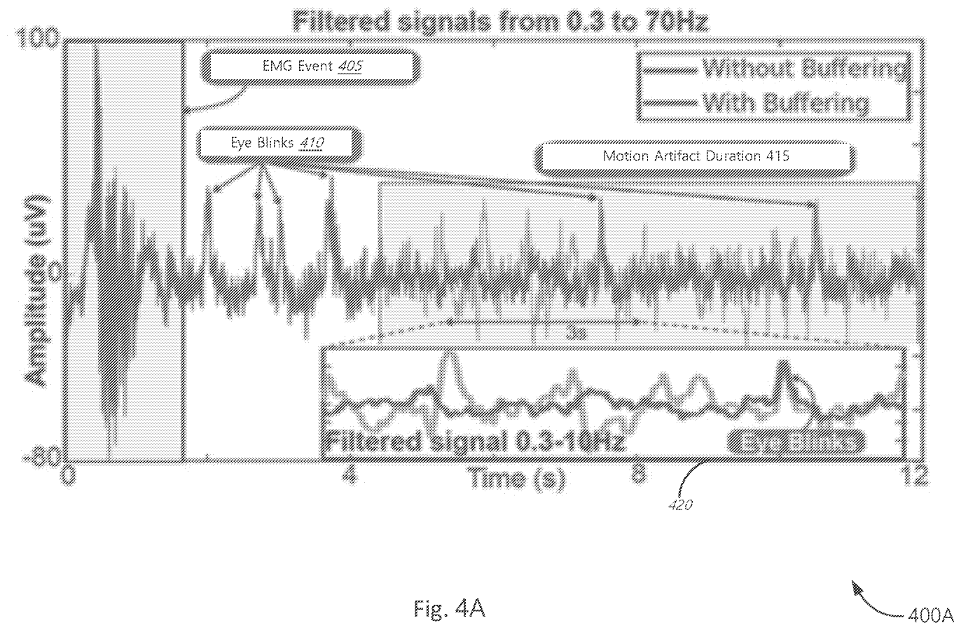

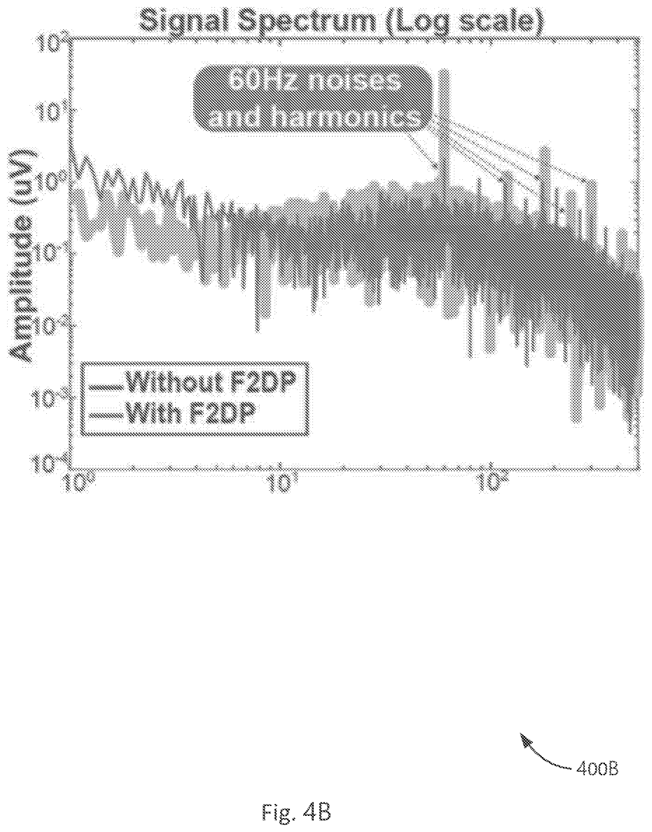

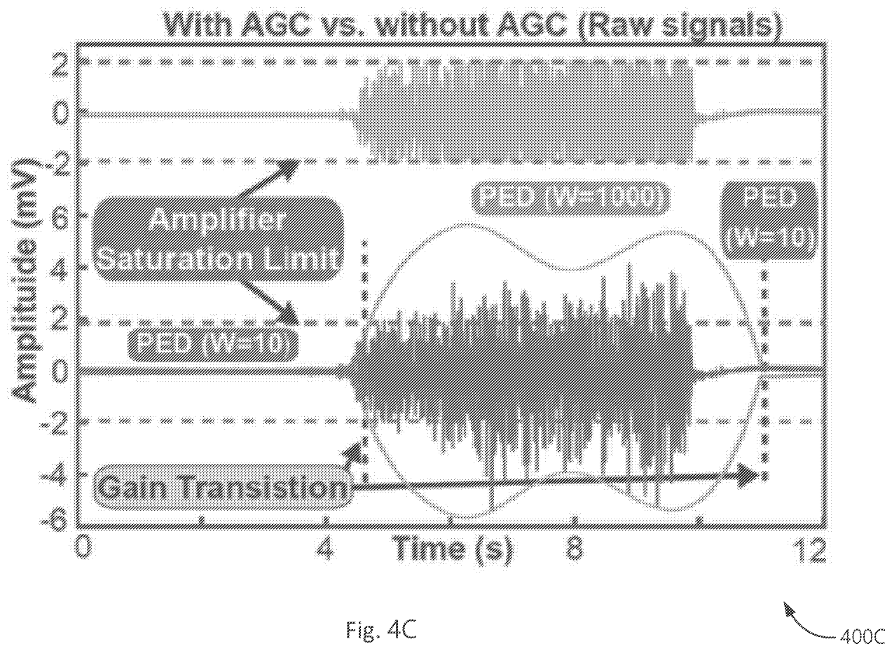

In various embodiments, the filter and amplifier 115 may be configured to filter the biosignals collected by the one or more sensors 120, and to amplify the signal for further processing by the signal conditioning logic of the sensing circuit 130 and BTE sensing logic 160. Accordingly, in various embodiments, the filter and amplifier 115 circuits may include on-board circuits housed within the one or more ear pieces 110. The filter and amplifier 115 may refer to a respective filtering circuit and amplifying circuit. In some embodiments, the filter and amplifier 115 may include low noise (including ultra low noise) filters and amplifiers for processing of the biosignals. In various embodiments, the filter and amplifier 115 may be part of a three-fold cascaded amplifying (3CA) circuit. The 3CA circuit may include a buffering stage, feed-forward differential pre-amplifying (F2DP) stage, and an adaptive amplifying stage, as will be described in greater detail below with respect to FIGS. 3-4C.

In various embodiments, the filter may include a high pass filter to remove DC components and only allow AC components to pass through. In some embodiments, the filter may be a Sallen-Key high pass filter (HPF). The 3CA circuit may include a buffering stage, F2DP stage, and adaptive amplifying stage, as described above. Thus, at the buffering stage, the amplifier may be configured to buffer a signal so as to minimize motion artifacts introduced by motion of the patient, including motion of the sensors and/or sensor leads and cables. In some embodiments, at the F2DP stage, the amplifier may be configured to amplify the one or more biosignals before driving the cables to the sensing circuit, thus a pre-amplification of the biosignals occurs at the F2DP stage. In some embodiments, gain of the pre-amplification stage may be chosen so as to utilize the full dynamic range of an analog to digital converter (ADC) of the sensing circuit. The adaptive amplification stage may be configured to compensate for significant amplitude range differences between various biosignals, such as between EEG/EOG signals and EMG signals. In some embodiments, the difference in amplitude range may cause signal saturation at an ADC of the sensing circuit. Thus, the amplifiers of the filter and amplifier 115 may comprise one or more different amplification circuits.

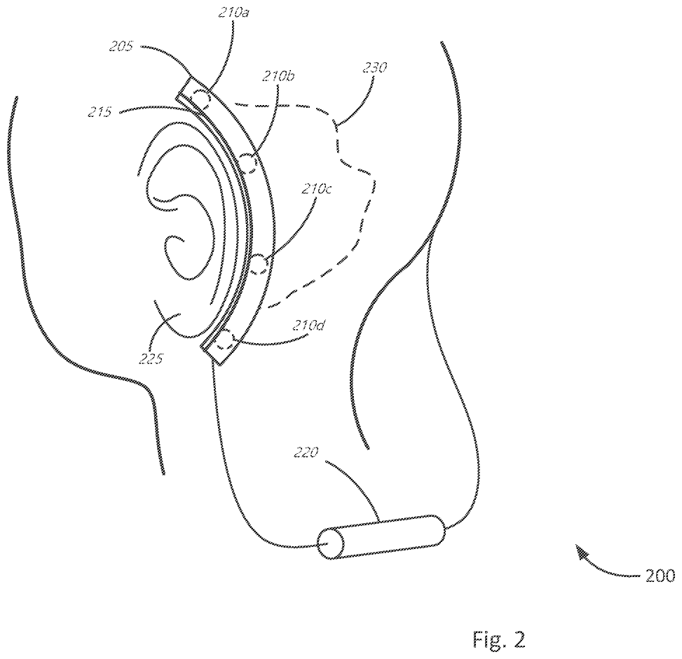

Once detected, and the one or more biosignals have been passed through the filter and amplifier 115 circuits, the biosignals may be fed to the sensing circuit 130 for further processing. The sensing circuit 130 may comprise, without limitation, an on-board processor to perform the on-board processing features described below. For example, the sensing circuit 130 may include, for example, an MCU, FPGA, custom IC or other embedded processor. In various embodiments, the sensing circuit 130 may be located, for example, in an in-line controller or other housing assembly in which the sensing circuit 130 and control logic for the one or more sensors 110 and the filter and amplifier 115 are housed (as will be described in greater detail below with respect to FIG. 2.

In various embodiments, the sensing circuit 130 may be configured to control sampling of the collected raw signals. For example, in some embodiments, the sensing circuit 130 may be configured to perform oversampling and averaging (OAA) 133 of the sensor signals (e.g., biosignals captured by the sensor), adaptive gain control (AGC) 131 of the adaptive amplifier circuit, and to manage wired and/or wireless communication and data streaming Thus, in various embodiments, the sensing circuit 130 may further include AGC logic 131, OAA logic 133, and streaming logic 140. In some further embodiments, the sensing circuit 130 may further comprise continuous impedance checking logic 135.

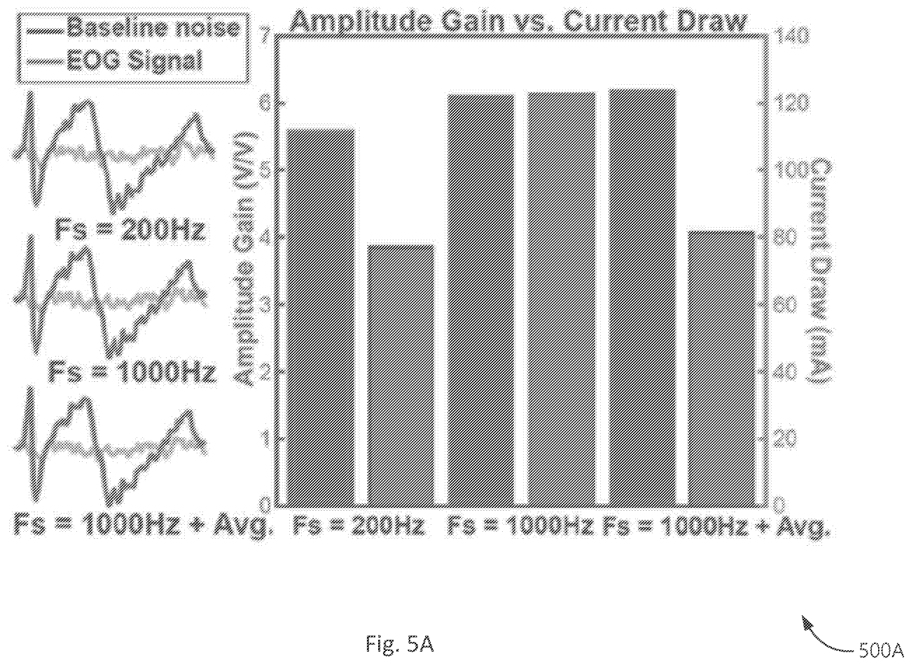

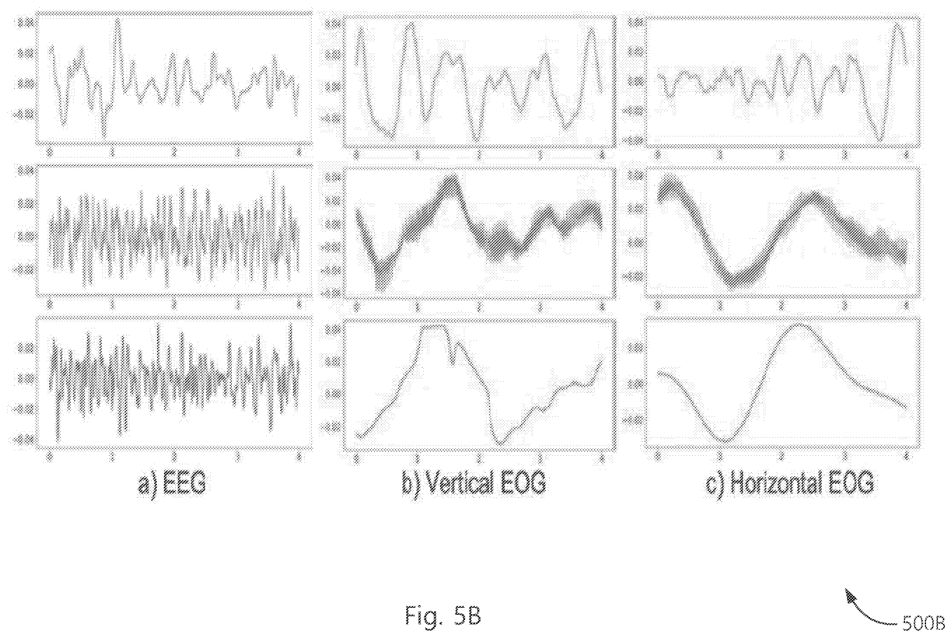

EEG, EOG, and EMG signals measured BTE are weaker than signals directly measured on the scalp, eyes, or chin. Thus, OAA 133 may be utilized to increase the signal-to-noise ratio (SNR) of measurements. For example, in some embodiments, the sensing circuit 130 may be configured to utilize a sampling rate of 1 kHz. Thus, to ensure high signal quality, the raw signal from the sensors may be oversampled at the 1 kHz sampling rate, and down-sampled by taking an average of the collected samples before being sent for further processing by the BTE sensing logic. Oversampling further aids in the reduction of random noises, such as thermal noise, variations in voltage supply, variations in reference voltages, and ADC quantization noises. Furthermore, OAA 133 may allow faster adjustments of the AGC 131 logic. In some embodiment, the oversampling rate of 1 kHz may be 10 times the frequency of interest. For example, in some embodiments, for microsleep detection, the maximum desired frequency for EEG, EOG, and EMG may be 100 Hz. Thus, in one example, to downsample the 1 kHz samples, an average may be taken for every 5 samples, producing a an output sampling rate of 200 Hz. FIG. 5A shows that by using OAA 133 with oversampling rate at 1 kHz and downsample to 200 Hz by averaging, a high SNR can be maintained while the total current draw of the sensing circuit is cut down.

After signals are collected from the device, they are further processed by the processing software 130. The processing software has two main components: (1) signals pre-processing, and (2) mixed-signals separation. All collected signals are cleaned and denoised by the signals pre-processing module by removing the 60 Hz electrical noise, direct current trend, spikes by notch, bandpass, median and outlier filters, respectively. Afterwards, the mixed-biosignals may be separated into EEG, EOG and EMG signals.

In some embodiments, the sensing circuit 130 may further comprise a driven right leg circuit (DRL) with a high common-mode rejection ratio configured to suppress noises coupling into the human body. Furthermore, the ground between the analog and digital components may be separated to avoid cross-talk interference.

Furthermore, in some embodiments, the sensing circuit 130 may be configured to perform continuous impedance checking for skin-electrode contact between the one or more sensors 120 and the skin of the patient 180. Thus, continuous impedance checking logic 135 may be configured to cause the sensing circuit to check, at a polling rate of 250 Hz, the impedance of a skin-electrode contact, to ensure any electrodes of the one or more sensors 120 maintain contact with the skin Thus, in some embodiments, a small electrical current (e.g., ˜6 nA), may be sent continuously through the contact between electrodes and the skin.

Contact impedance is measured by sending in an AC signals at the frequency of interest. Typically, 30 Hz is usually chosen for EEG measurement. This, however, will create noises to the measurement so it can't be used in the middle of a recording without stopping it. To avoid unnecessarily stopping recording, in at least one embodiment, the system measures impedance at a higher frequency than the frequency of interest because the impedance values are linearly proportional to the frequency in the log scale in the range from 10 to 1000 Hz. Thus, in at least one embodiment, the system measures the contact impedance at 250 Hz which is much higher than our frequency of interest (i.e. 0.1 Hz to 100 Hz). As a result, contact quality of electrodes can be continuously monitored.

In some embodiments, the sensing circuit 130 may further include the adaptive amplifying circuit described above, while in other embodiments, the adaptive amplifying circuit may be part of the filter and amplifier 115 circuits. Accordingly, in some embodiments, the on-board processor of the sensing circuit 130 may further be configured to dynamically adjust the gain of the adaptive amplifier to ensure that biosignals, such as the smaller amplitude EEG and EOG signals, and larger amplitude EMG signals are captured with high resolution. In some embodiments, the adaptive amplifier may have an adjustable gain range of 1× to 24× gain.

In some embodiments, the difference between the smaller amplitude EEG and EOG signals, and larger amplitude EMG signals may be on the order of 1000×. Thus, the analog gain in the sensing circuit 130 may be dynamically adjusted to adapt to the changes in signal amplitude. In some embodiments, the AGC 131 logic may accordingly be configured to dynamically adjust the gain of the adaptive amplifier circuit according to an AGC algorithm. In some embodiments, the AGC algorithm may be configured to adjust gain according to EEG/EOG signals and EMG events. For example, when no significant EMG events are occurring, the EEG/EOG signals may be kept as maximum gain. The AGC 131 logic may further be configured to react quickly to abrupt increases in amplitude of EMG events (e.g., quickly attenuate an EMG signal), and react slowly to decreases in amplitude while an EMG event is ongoing to avoid gain oscillation. In some embodiments, AGC techniques, such as a peak envelope detector or square law detector may be employed. In one example, utilizing a peak envelope detector, a small window may be utilized while no EMG events occur such that the peak envelope detector can react quickly to increases amplitude, while a large window may be utilized while an EMG event is occurring so as to avoid gain oscillations in some embodiments, the window size of the lower and upper threshold may be set at 128 samples, and 70% and 90% maximum range of the current gain value, respectively. During a gain transition, several samples may be lost because the amplifier needs to be stabilized before new measurements can be done. Thus, missing samples may be filled using linear interpolation techniques.

In various embodiments, AGC may be implemented after OAA (e.g., after downsampling). In other embodiments, AGC 131 logic may be implemented after oversampling, but before averaging (e.g., downsampling).

According to various embodiments, the gain may be changed adaptively instead of having a fixed value. In at least one embodiment, AGC overcomes at least two problems in the art. (1) AGC solves the problem of signal saturation where the amplitude of the DC (direct current) part of the captured signal varies reaches the maximum dynamic range of the ADC resulting in saturation and no signals being captured. By adaptively changing the gain and adjust the dynamic range of the ADC, saturation can be avoided. (2) AGC also increase fidelity of the captured signals. Small signals like brainwaves (i.e EEG), which can be as small as 10 uV, are a thousand times smaller than large signals such as muscle contractions, which can be as strong as 100000 uV. Thus, the AGC may be configured to increase the gain for getting small signals with high resolution while flexibly and quickly decreasing gain to capture large signals.

In various embodiments, the on-board processor of the sensing circuit 130 may further be configured drive the analog front end (AFE) on the sensing circuit 130 to collect EEG/EOG/EMG and EDA measurements from the one or more sensors 120, adjust the amplifier gain dynamically to ensure high resolution capture of weak amplitude signals while preventing strong amplitude signals from saturation, and to manage communication to a host device in various embodiments, the signal conditioning logic (e.g., AGC 131 logic, OAA logic 133) of the sensing circuit 130, as well as continuous impedance checking logic 135 and streaming logic 140, may include computer readable instructions in a firmware/software implementation, while in other embodiments, custom hardware, such as dedicated ICs and appliances may be used.

In various embodiments, the sensing circuit 130 may further include streaming logic 140 executable by the processor to communicate with a host machine 150 and/or stream data to on-board local storage, such as on-board non-volatile storage (e.g., flash, solid-state, or other storage) Accordingly, in some embodiments, the BTE device 105 and/or sensing circuit 130 may include a wireless communication chipset (including radio/modem), such as a Bluetooth chipset and/or Wi-Fi chipset. In yet further embodiments, the BTE device 105 and/or sensing circuit 130 may further include a wired communication chipset, such as a PHY transceiver chipset for serial (e.g., universal serial bus (USB) communication, Ethernet, or fiber optic communication). Thus, the streaming logic 140 may be configured to manage both wireless and/or wired communication to a host machine 150, and in further embodiments to manage data streaming to on-board local storage.

In various embodiments, host machine 150 may be a user device coupled to the BTE device 105. For example, the host machine 150 may include, without limitation, a desktop computer, laptop computer, tablet computer, server computer, smartphone, or other computing device. The host machine 150 may further include a physical machine, or one or more respective virtual machine instances. The host machine 150 may include one or more processors 155, and BTE sensing logic 160. BTE sensing logic 160 may include, without limitation, software (including firmware), hardware, or both hardware and software. The BTE sensing logic 160 may further include signal processing logic 165, signal separation logic 170, and wakefulness level classification logic 175.

In various embodiments, the host machine 150 may be configured to obtain the biosignal from the BTE device 105 after pre-processing (e.g., OAA and AGC) of the signal. Once received, the signal from the BTE device 105 may include one or more biosignals (e.g., EEG, EOG, EMG, and EDA), which are combined into a single signal. Thus, the biosignal received from the BTE device may be a mixed signal comprising one or more component biosignals. In various embodiments, the signal processing logic 165 may be configured to apply a notch filter to all sensor data to remove 50/60 Hz power line interference, linear trend removal to avoid DC drift, and outlier filters to remove spikes and ripples.

In various embodiments, signal separation logic 170 may be configured to separate teach component biosignal from the mixed signal received from the BTE. As discussed above, the mixed signal may include EEG, EOG, and EMG data. In various embodiments, EEG data may be defined at the frequency range of 4-35 Hz, EOG defined at a frequency range of 0.1-10 Hz, and EMG at a frequency range of 10-100 Hz Thus, the signal separation logic 170 may be configured to separate each component biosignal data from the mixed signal received from the BTE device 105. In some embodiments, respective bandpass filters (BPF) may be applied to split the overlapping BTE biosignals into signals at the respective frequency ranges of interest. In some embodiments, wakefulness-related EEG bands (e.g., θ, α, and β waves) using 4-8 Hz, 8-12 Hz, and 12-35 Hz BPFs, respectively. Horizontal EOG (hEOG) for eye movement and vertical EOG (vEOG) for eye blinks may be extracted using a 0.3-10 Hz BPF. A 10-100 Hz BPF and a median filter may then be applied to the mixed signal to extract EMG band while removing spikes and other excessive components.

In various embodiments, an EDA signal may further be extracted by the signal separation logic 170. EDA may be a superposition of two different components: skin conductance response (SCR) and skin conductance level (SCL) at the frequency range of 0.05-1.5 Hz and 0-0.05 Hz, respectively. After removal of common noises, BPFs may similarly be applied to extract low-frequency components of the EDA signal. In some embodiments, a polynomial fit of the original EDA signal may be added back before linear trend removal with the output of the 0.05-1.5 Hz BPF to obtain the complete SCR component signal of the EDA signal.

In various embodiments, wakefulness classification logic 175 may include logic for wakefulness feature extraction from one or more of the component biosignals for EOG, EEG, EMG, and EDA. Furthermore, the wakefulness classification logic 175 may employ a machine learning (ML) model to determine a wakefulness level of the patient 180 based on the biosignals. As will be described in greater detail below, with respect to FIG. 1B, various temporal, spectral, and non-linear features may be identified and extracted from the respective component biosignals, and the ML model may classify a wakefulness level of the patient 180. In some embodiments, the wakefulness level may be a normalized scale configured to quantify a wakefulness of the patient. In one example, the wakefulness level may be normalized on a scale from 0 to 1, where 0 indicates a state of sleep or microsleep, and 1 indicates an awake state. In some embodiments, the scale may indicate an estimated probability that the patient is in a sleep and/or microsleep state, with 0 being 100% certainty, and 1 being 0% certainty. Alternatively, the ML model may be configured to classify whether the patient 180 is in an awake state, sleep state, and/or in a state of microsleep. Accordingly, in some embodiments, the wakefulness classification may be used in association with one or more of the following applications narcolepsy sleep attack warning, continuous focus supervising, driving/working distraction notification, and epilepsy monitoring.

The BTE sensing logic 160 may, in some embodiments, be configured to control the stimulation output 125 based on the wakefulness classification (e.g., microsleep classification) determined above. Alternatively, in further embodiments, the host device 150 may be configured to transmit the wakefulness/microsleep classification to the on-board processor of the BTE device 105, which may in turn be configured to control stimulation output 125 based on the wakefulness classification. For example, if microsleep is detected by the BTE sensing logic 160, the BTE device 105 and/or BTE sensing logic 160 may cause the stimulation output 125 to be activated. Accordingly, in various embodiments, the stimulation output 125 may be configured to provide various forms of stimulation, and therefore may include one or more different types of stimulation devices. For example, the stimulation output of the BTE device 105 may include, without limitation, a light source, such as a light emitting diode or laser, speakers, such as a bone conductance speaker or other acoustic speaker, electrodes, antennas, magnetic coils, among other stimulation.

In some embodiments, the stimulation output 125 may be incorporated into one or more of the one or more ear pieces 110. For example, the stimulation output 125 may be integrated into the one or more earpieces 120, such as an array of electrodes, bone conductance speakers, antennas, light emitters, and various other similar components in some embodiments, RF and EM stimulation from the antennas, electrodes, and/or magnetic coils of the stimulation output 125 may be directed to desired parts of the brain utilizing beamforming or phase array techniques. Additionally, acoustic and ultra-sound signals can be produced through a bone conductance speaker to transmit audio instruction to the patient 180, or to provide auditory stimulation to the patient 180, such as an alarm sound. In further embodiments, the light source may be an adjustable frequency light source, such as a variable frequency LED or other laser Thus, the frequency of the light source may be changed. Moreover, the light source may be positioned to provide light stimulation to the eyes, or to expose skin around the head to different frequencies of light.

In yet further embodiments, as previously described, the stimulation output 125 may be incorporated, at least in part, in a separate stimulation assembly. The stimulation assembly may include, for example, and without limitation, a mask, goggles, glasses, cap, hat, visor, helmet, or headband Thus, the stimulation output 125 may be directed towards other parts of the head in addition to areas in contact with the BTE device 105/one or more ear pieces 110. For example, the stimulation output 125 may be configured to direct light towards the eyes of the patient 180. In some embodiments, light may be directed to other parts of the face of the patient 180. In some embodiments, electrical stimulation may be provided through the scalp of the patient, or on different areas of the face, neck, or head of the patient. In yet further embodiments, audio stimulation may be provided via headphone speakers, or in-ear speakers.

FIG. 1B is a functional block diagram of a behind-the-ear sensing and stimulation system 100B, in accordance with various embodiments. The BTE system 100B includes signal processing logic 165, which includes DC removal 165a, notch filter 165b, median and outlier filter 165c, and denoise 165d. The system further includes signal separation logic 170, which includes BPF 170a, ground-truth signal 170b, transfer learning function 170c, EOG signal 171a, EEG signal 171b, EMG signal 171c, and EDA signal 171d wakefulness classification logic 175, which includes wakefulness feature extraction 175a, machine learning model 175b, and wakefulness level 175c, and sensor data stream 185, which includes mixed EEG, EOG, and EMG signals 185a, EDA signal 185b, motion/positional signal 185c and contact impedance (Z) signal 185d. It should be noted that the various components of the system 100A are schematically illustrated in FIG. 1A, and that modifications to the system 100A may be possible in accordance with various embodiments.

In various embodiments, a sensor data stream 185 may be sensor data obtained from the BTE device. Thus, the sensor data stream 185 may include mixed EEG, EOG, and EMG signal 185a (also referred to as the “mixed signal 185a), EDA signal 185b, motion/positional signal 185c, and contact Z signal 185d. The mixed signal 185a and EDA signal 18 db may be transmitted to a host device for further signal processing via signal processing logic 165. Signal processing logic includes DC removal 165a, notch filter 165b, median an outlier filter 165c, and denoise 165d. Motion/positional signal 185c and contact Z signal 185d may also be obtained, from the BTE device, for denoising of the mixed signal 185a and EDA signal 185b. The signal, once processed, may be separated into component biosignals by signal separation logic 170. Signal separation logic 170 includes BPF logic 170a, and transfer learning function 170c. Ground-truth signal 170b may further be used by the transfer learning function 170c to train the transfer learning algorithm. The transfer learning function 170c may output the separated EOG signal 171a, EEG signal 171b, and EMG signals 171c. The separated biosignals may then be provided to the wakefulness classification logic 175, which may include wakefulness feature extraction 175a, ML model 175b, and wakefulness level 175c. The determined wakefulness level 175c may then be fed back to the BTE device in sensor data stream 185.

Accordingly, as discussed above, sensor data stream 185 may include the data stream obtained by a host machine 150/sensing logic 160 from the BTE device 105. Thus, sensor data stream 185 includes pre-processed (e.g., OAA, filtered and amplified) signals obtained by the host machine 150 from the BTE device 105. The sensor data stream 185 may include the mixed signal 185a, EDA signal 185b, motion/positional signal 185c, and contact impedance signal 185d. The mixed EEG signal 185a and EDA signal 185b may then be processed by the signal processing logic 165.

As previously described, signal processing logic 165 may include DC removal 165a. For example, as previously described, a HPF may be applied to remove any DC components. Moreover, in some embodiments, DC removal 165a may include linear trend removal to avoid DC drift. In some embodiments, a notch filter 165b may be applied to remove 50/60 Hz power line interference, and a median and outlier filter 165c may be applied to remove spikes and ripples. The signal processing logic 165 may, in some embodiments, then calculate a linear fit to the mixed biosignal. The linear fit may comprise a sextic fit.

Regarding the EDA signal 185b, as previously described, the EDA signal 185b is the superposition of two different components, SCR and SCL at the frequency range of 0.05-1.5 Hz and 0-0.05 Hz, respectively. SCL can vary substantially between individuals, but SCR features reflect a fast change or reaction to a single stimulus give the general characteristics of EDA signal. After removing common noises as mentioned above using the the notch filters 165b, and median and outlier filters 165c, BPF function 170a may apply respective BPFs to extract the low-frequency and meaningful components of the EDA signal. In at least one embodiment, in order to extract full information from the SCR, the system adds back the sextic polynomial fit of the EDA signal 171d with the output of bandpass 0.05 to 1.5 Hz filter to get the complete SCR component.

As previously described, in order to ensure that electrical contacts with the skin remain strong, contact Z signal 185d may continuously be captured at the electrode-skin contact point. In some embodiments, a patient may be notified to adjust the BTE device 105 and/or one or more ear pieces 110 to maintain good contact. In some embodiments, the contact Z signal 185d may further be used to mitigate the effects of patient 180 movement. In some embodiments, a sinusoidal excitation signal may be used, with a frequency of 250 Hz. Thus, a BPF from 248-252 Hz may be applied, and an RMS envelope taken to calculate impedance values: Z(ω)=Vᵣₘₛ*\sqrt{I}.

In some embodiments, denoise function 165d may further denoise the processed signal from any noise introduced by movement or caused by inadequate electrode contact with the skin of the patient. Accordingly, the denoise function 165d may be configured to denoise the signal based, at least in part, on the motion/positional signal 185c and contact Z signal 185d obtained from the BTE device 105. The motion/positional signal 185c may include, without limitation, signals received from motion and/or positional sensors of the BTE device 105 and/or on the one or more ear pieces 110. Motion/positional signal 185c may include signals received, for example, from one or more of an accelerometer, gyroscope, IMU, or other positional sensor, such as a GNSS receiver.

In various embodiments, pre-processing techniques may be applied to the motion/positional signal 185c (i.e Kalman filter, DCM algorithm, and complementary filter). In at least one embodiment, motion/positional signal 185c may be pre-processed at the BTE device 105 and/or host machine 150. In one example, motion/positional signal 185c may have complementary filters applied due to the simple filtering calculation and lightweight implementation. In at least one embodiment, the complementary filter takes slow moving signals from an accelerometer and fast moving signals from a gyroscope and combines them. The accelerometer may provide an indication of orientation in static conditions, while the gyroscope provides an indication of tilt in dynamic conditions. The signals from the accelerometer may then be passed through a low-pass filter (LPF) and the gyroscope signals may be passed through an HPF, and subsequently combined to give the final rate.

In further embodiments, motion/positional signal 185c may further be pre-processed using the spikes removal approach. The data from 3 sensors may be fused based on the complementary filtered signal, given as α=0.98*(α+g*dt)+0.02*α, where α is an angular position. The denoise function 165d may then estimate the power at the angular position in order to mitigate noise and artifacts introduced by the movement of the patient 180.

The denoised signal from the denoise function 165d may then be used by signal separation logic 170 to separate the mixed signal into its component biosignals. Denoise information from the denoise function 165d may further be provided to the wakefulness feature extraction logic 175a to distinguish identified features from motion artifacts and/or artifacts introduced by electrode contact issues.

Signal separation logic 170 may include BPF function 170a, which may apply one or more BPFs to the mixed signal to separate the component signals. As previously described, EEG data may be defined at the frequency range of 4-35 Hz, EOG defined at a frequency range of 0.1-10 Hz, and EMG at a frequency range of 10-100 Hz. Thus, BPF functio1 70a may apply one or more respective BPFs at the corresponding frequency ranges to extract the individual component biosignals of EOG 171a, EEG 171b, and EMG 171c.

As discussed above, since EEG, EOG, EMG have overlapping frequencies in the mixed signal 185a, the signal separation logic 170 may be configured to separate the mixed signal 185a into individual EEG, EOG, and EMG signals by combining both conventional bandpass filtering and utilizing transfer learning in at least one embodiment, the transfer learning model is built based on general ‘ground-truth’ signals which helps to eliminate of per-user training. The general ground-truth signals may be gathered from one or more individuals using hospital-grade sensors.

In at least one embodiment, and as discussed above, different band-pass filters extract various biosignals considering the frequency bandwidth of each biosignal. Further, the system may selectively determine what features are important from the biosignal based upon the desired outputs. For example if the system is determining wakefulness with respect to EEG signals, among five waves of the EEG signal, the may ignore the δ waves (associated with deep sleep) and γ waves (heightened perception) because the system is focused on wakefulness and drowsiness. The BPF function may then extracts the θ, α, and β-waves by applying 4-8 Hz, 8-13 Hz, 13-35 Hz BPFs, respectively. After signal separation, in some embodiments, the signal processing logic 165 may again apply a median filter 165c to smooth out the signals.

Similarly, as previously described, when processing the EOG signals, the signal separation logic 170 may separate the EOG signal into horizontal EOG (hEOG) for eye movement and vertical EOG (vEOG) for eye blink. Thus, BPFs may be applied, at the BPF function 170a, to separate the hEOG and vEOG signals at 0.2-0.6 Hz and 0.1-20 Hz, respectively. Thus, EOG signal 171a may include individually separate vEOG and hEOG signals. The signal processing logic may then, again, apply a median filter 165c to clean the EOG signals. For EMG signals, a 50-100 Hz BPF may be applied, which is the dominant frequency range, and a median filter 165c applied to get rid of spikes and other excessive components.

In some further embodiments, a combination of BPF function 170b and transfer learning may be utilized to separate the component biosignals. For example, in some embodiments, once the one or more respective BPFs have been applied as described above, the resulting signals may still have overlapping frequency components Thus, a deep learning model similar to a convolutional autoencoder may be utilized to separate the respective component biosignals in the overlapping frequency ranges, as described in greater detail below.

In some embodiments, the convolutional autoencoder may learn the pattern of each class of signal, i.e. EEG, EOG and EMG, from the signals captured by a ground-truth signal 170b. In some embodiments, the ground-truth signal 170b may be obtained from an existing source or database. In some embodiments, the ground-truth signal 170b may, for example, include data obtained from a “gold-standard” PSG recording device at the places where the signals are generated such as the scalp (EEG), the eyes (EOG) and the chin (EMG) The ground-truth signal 170b may include measurements taken from the patient 180, or from other individuals. Accordingly, the ground-truth signal 170b may or may not be from the same person. Thus, in some examples, the ML model may learn a general pattern from data taken from a test population of individuals.

Experimental results have shown that combined BPF function 170a and transfer learning function 170c result in improved signals that are 50% closer to the ground-truth signals captured directly from the patient 180 (e.g., via a PSG recording device) in terms of Euclidian distance. FIG. 5B illustrates graphs 500B depicting EEG and EOG signals obtained after bandpass filtering of EEG frequency (top row), reconstructed EEG and EOG signals by a transfer learning function 170c (middle row), and ground-truth EEG and EOG signals captured by a PSG recording device with electrode placement on the scalp (bottom row).

In at least one embodiment, to further extract information from the biosignals, the system may utilize a deep learning model similar to a convolutional autoencoder Convolutional autoencoders comprise a network of stacked convolutional layers that take as input some high dimensional data point and place that point in a learned embedded space. Autoencoders usually embed the data into a space with fewer dimensions than the original input with applications in data compression or as a preprocessing step for other analyses that are sensitive to dimensionality. Normally, to train such an autoencoder requires a mirrored stack of decoding convolutional layers that attempt to inverse the transformation of the encoding layers, with the update value based on the distance between the decoded output and the original input data. However, in at least one embodiment, the system instead trains the model based on its ability to extract one of the physiological signals from the mixed signal input directly. Therefore, the model only requires encoding layers, with the embedded space being the signals drawn from the distribution of the given physiological signals.

Convolutional layers are a specialized type of neural network developed for use in image classification that apply a sliding kernel function across the data to extract primitive features. Additional convolution layers can be applied to the primitive features to learn more abstract features that are more beneficial to classification than the original input. In at least one embodiment, convolution can apply just as readily to a single dimension, such as along a time series, to extract similar features and, depending on how the layers are sized, can learn to extract features more related to either temporal or spectral features. Accordingly, in at least one embodiment, the model extracts both temporal and spectral features by first training a model to detect temporal features, then another to detect spectral features, and finally transferring the weights from those models to a combined model to finetune the learning of the combination of features. Splitting the model for pretraining reduces the amount of learning each half of the model is responsible for which helps with convergence, and produces a stronger separation in the end. Another advantage of this method of training is that, for signals with either stronger temporal or spectral features, one side of the model can be weighted over the other or dropped entirely in favor of the stronger features, for example with EOG signal separation.

In various embodiments, after obtaining individual EEG 171b, EOG 171a (including individual hEOG and vEOG signals), EMG 171c, and EDA 171d signals, wakefulness classification logic 175 may be configured to segment time series data from each of the individual signals into fixed-size epochs, and selected features extracted from each epoch. Thus, in various embodiments, wakefuleness feature extraction 175a may include the selection and identification of features. In some examples, features may include temporal features, spectral features, and non-linear features.

In various embodiments, temporal features may be features used in time-series data analysis in the temporal domain. For example, temporal features may include, without limitation, mean, variance, min, max, hjorth parameters, skewness, and kurtosis. As EOG 171a, EMG 171c, and EDA 171d signals are often analyzed in the time domain, in some embodiments, one or more of the temporal features may be extracted for each of hEOG, vEOG, EMG, and EDA. In some examples, wavelet decomposition may be used for the hEOG signal to extract saccade features—specifically mean velocity, maximum velocity, mean acceleration, maximum acceleration, and range amplitude Eye blink features, namely, blink amplitude, mean amplitudes, peak closing velocity, peak opening velocity, mean closing velocity and closing time are extracted from vEOG signal.

In various embodiments, spectral features may be features used in data analysis in the frequency domain. For example, spectral features may include, without limitation, ratio of powers, absolute powers, θ/β, α/β, θ/α, and θ/(β+α). Spectral features of the EEG signal 171b may be analyzed as brainwaves are generally available in discrete frequency ranges at different stages. Thus, one or more of the spectral features may be extracted for each of the two channels (e.g., left ear and right ear) of EEG signal 171b.

In various embodiments, non-linear features may be features showing complex behaviors with nonlinear properties. Non-linear features may include, without limitation, correlation dimension, Lyapunov exponent, entropy, and fractional dimension. Non-linear analysis may be applied to the chaotic parameters of the EEG signal 171b. Thus, one or more of the non-linear features may be extracted for each of the two channels of EEG signal 171b.

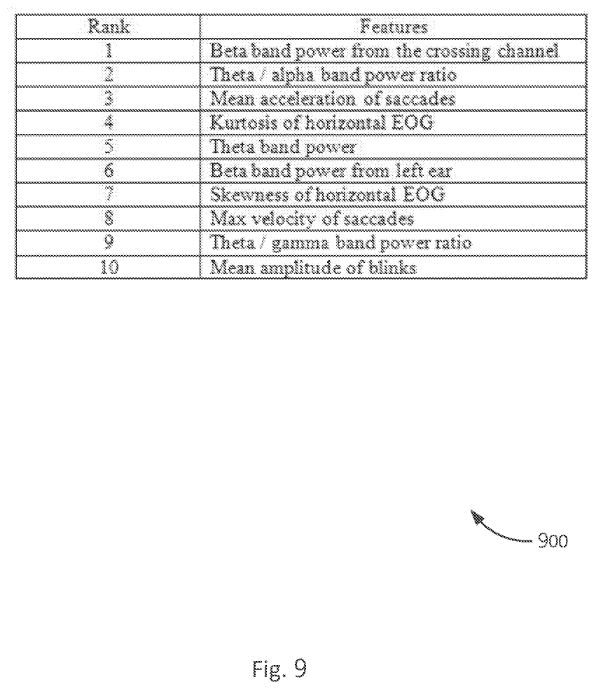

FIG. 9 is a table 900 of features that may be extracted from the various separate biosignals. The table 900 comprises features that have been determined to be significant to quantify wakefulness levels identified by the ML model 175b. When all possible features are used altogether, irrelevant correlated features or feature redundancy may degrade the performance. Thus, in some embodiments, feature selection techniques may be applied by wakefulness feature extraction 175a Feature selection methods may include, without limitation, Recursive Feature Elimination (RFE), L1-based, and tree-based feature selection. In some examples, RFE is a greedy optimization algorithm that removes the features whose deletion will have the least effect on training error. L1-based feature selection may be used for linear models, including Logistic Regression (LR) and support vector machine (SVM). In some embodiments, L1 norms may be used in a linear model to remove features with zero coefficients. In further embodiments, a tree-based model may be used to rank feature importance and to eliminate irrelevant features.

In various embodiments, the ML model 175b may be configured to determine the most relevant features to a wakefulness classification 175c, and to determine a wakefulness classification 175c based on the identified features. Various classification methods may be utilized by the ML model 175b, including, without limitation, SVM, linear discriminant analysis (LDA), LR, decision tree (DT), RandomForest and AdaBoost. In some embodiments, a hierarchical stack of 3 base classifiers may be utilized by the ML model 175b. In some examples, the stack may comprise RandomForest classifier (with 50 estimators) in the first layer, Adaboost classifier (with 50 estimators) in the second layer, and SVM (with RBF kernel) in the last layer.

Continuing with the above example, in some embodiments, for the first two layers (RandomForest and Adaboost), only the predictions with high probabilities (greater than 0.7) may be kept, and the remaining samples may be transferred to the subsequent layer. In the last layer, SVM may classify all of the remaining samples. In some further embodiments, a heuristic classification rule may be applied, by the ML model 175b, to the final prediction based on knowledge that an EMG event is highly likely to lead to an “awake” event.

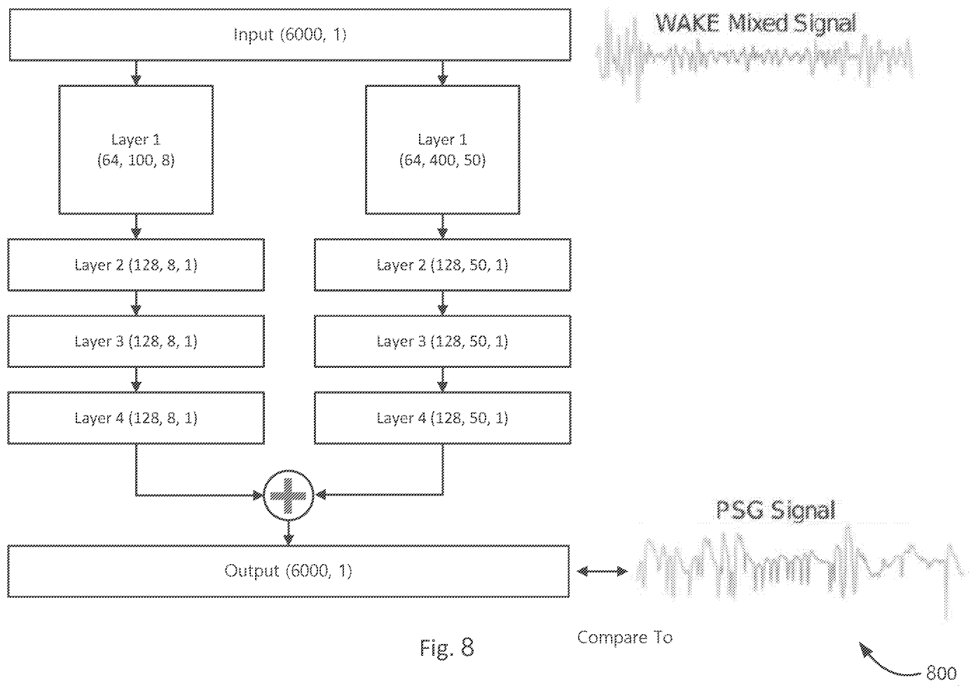

FIG. 8 is a schematic representation of the machine learning model 175b, 800. The depicted model 800 shows each convolutional layer specified by a tuple of (kernel size, number of filters, stride size). The temporal and spectral feature separators are represented by the left and right tracks of the model respectively. Temporal features are extracted using a low-parameter and small stride kernel that can identify longer, more simple trends and spectral features using a high-parameter, large stride kernel to learn more complex spectral features without learning overlapping trends. In some examples, each model may be trained using logcosh as the loss distance function, which is similar to mean squared error, but less sensitive to large differences in outliers, and the Adam optimizer with a learning rate of 0.01.

Models may be trained for 100 epochs on each signal type with shuffled 30 s epochs from all subjects, then used to predict the separated signal for each subject with each 30 s epoch in chronological order. Once both temporal and spectral separators have been trained, the weights from each are transferred to the combined model that adds the outputs of the two before comparison to the target signal. The combined learner uses the same loss and optimizer but with a reduced learning rate of 10E−6. Before use in the model, both the signals are normalized such that each 30 s epoch has a mean of 0 and a standard deviation of 1, signals are all aligned in time between the PSG and the wakefulness signal and the wakefulness signal is down sampled to 200 hz to match the PSG signal sampling rate.

Thus, in various embodiments, the ML model 175b may classify a user's wakefulness level 175c based on features identified and extracted, such as those listed in table 900 of FIG. 9. In at least one embodiment, a hierarchical classification model may be built with these significant features, and can achieve 76% precision and 85% recall on an unseen subject. The results of wakefulness levels 175c may be used to produce the feedback to the patient 180 by using acoustic signals to warn the user, or to provide stimulation to the patient via the stimulation output 125, and further providing feedback of the effects of the stimulation through sensor data stream 185.

FIG. 2 is a schematic perspective view diagram of a BTE device 200 worn by a patient. The BTE device 200 includes ear pieces 205 including a left ear piece (shown) and a right ear piece (obscured from view), one or more sensors 210 (including a first sensor 210a, second sensor 210b, third sensor 210c, and fourth sensor 210d), memory wire 215, in-line controller 220. It should be noted that the various components of the BTE device 200 are schematically illustrated in FIG. 2, and that modifications to the BTE device 200 may be possible in accordance with various embodiments.

In various embodiments, each of the one or more ear pieces 205 may comprise one or more sensors 210. In some embodiments, the one or more sensors 210 may include a first sensor 210a, second sensor 210b, third sensor 210c, and fourth sensor 210d. Each respective ear piece 205 may further comprise a memory wire 25 configured to be manipulated into a desired shape, and to maintain a configuration and/or shape into which it is manipulated. The one or more sensors 210 may further be coupled to the in-line controller 220, which may further comprise on-board processing, storage, and a wireless and/or wired communication subsystem.

As previously described, the one or more ear pieces 205 may be a silicone or other polymeric material. The one or more ear pieces 205 may be configured to be worn around the ear 225 in a BTE configuration. Thus, the one or more sensors 210 may be in contact with skin BTE of the patient. The one or more ear pieces 205 may be configured to conform to the shape of the mastoid bone 230 located behind the ears of the patient, and the one or more sensors to be in contact with the skin over the mastoid bone 230. In some embodiments, to better conform to the curve of the mastoid bone, the one or more sensors 210 may comprise an array of electrode sensors coupled to the skin of the patient, for example, via an adhesive or other medium. In other embodiments, the electrode sensors may be fixed in position via the body of the ear piece 205, which may, in some examples, similarly include an adhesive or other contact medium.

In some embodiments, the one or more ear pieces 205 may comprise silicone or other polymeric material that is pliable so as to conform to the curve behind the ear 225 of the patient. In some embodiments, the ear piece 205 may be molded based on the average size of the human ear (the average ear is around 6.3 centimeters long and the average ear lobe is 1.88 cm long and 1.96 cm wide). To maintain the good contact between the electrodes and the skin, the earpieces 200 may comprise a memory wire 215 inside the body of the ear piece. The memory wire 215 may aid in pressing the respective ear pieces 205, and in turn the one or more sensors 210 against the skin of the patient and further to allow the ear piece 205 to stay in place around the ear 225 of the patient. Similarly, as previously described, the one or more sensors 210 may be made from one or more of silver fabric, copper pad, gold-plated copper pad material. In further embodiments, an electrode of the one or more sensors 210 may be formed from hard gold to better resist skin oil and sweat.

In at least one embodiment, various additional components can be integrated into the one or more ear pieces 205. For example, each of the one or more ear pieces 205 may further comprise stimulation outputs (e.g., components to provide stimulation to the patient). Stimulation outputs may include, for example, a light source, such as a light emitting diode or laser, speakers, such as a bone conductance speaker or other acoustic speaker, electrodes, antennas, magnetic coils, among other stimulation components, as previously described.

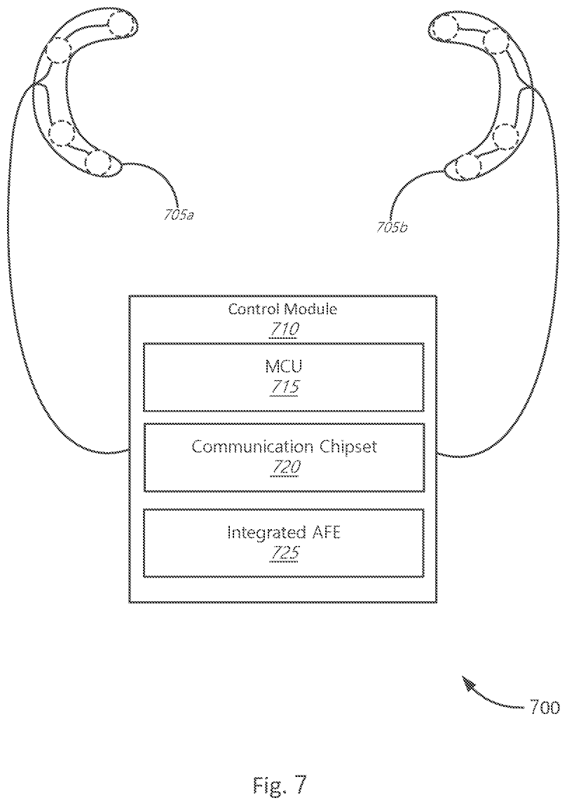

In various embodiments, the in-line controller 220 may comprise an on-board processor, storage, and communication subsystems. The in-line controller 220 may further include a sensing circuit configured to capture and digitize analog biosignals captured by the one or more sensors 210. On-board processing logic in the in-line controller 220 may further be configured to pre-process the biosignals, as previously described. The in-line controller 220 may further be configured to stream data that has been pre-processed to on-board storage and/or to establish a wired and/or wireless connection to a host computer for further processing and wakefulness classification, as described above. Hardware implementations of the in-line controller are described in greater detail below with respect to FIG. 7.

In many cases, even with good contact, captured bio-signals from an earpiece are weak because the electrodes are placed behind the ear and far away from the signal sources (i.e. the brain, the eyes and the facial muscles) compared to placing them on the scalp. Accordingly, in at least one embodiment, a high sampling rate is utilized to increase signal-to-noise ratio (SNR) within the sensor system Additionally, BTE signals are unique because the EEG, EOG and EMG signals are mixed together, and the system needs to be able to extract them. Furthermore, each type of signal has different amplitude ranges. The EMG signal could be as large as 100 mV while the EEG and the EOG could be as small as 10 uV, which is 10,000 times smaller. Further, electrical measurements of EEG, EOG and EMG are usually affected by motion artifacts. These motion artifacts need to be mitigated in order to maximize resulting clean signals.

Thus, FIG. 3 is a schematic circuit diagram of 3CA active electrode circuit 300 (also referred to interchangeably as the “3CA circuit”), in accordance with various embodiments. The 3CA active electrode circuit 300 includes a buffering stage 350, F2DP stage 355, and adaptive amplifying stage 360. It should be noted that the various components 3CA circuit arrangement of the active electrode circuit 300 are schematically illustrated in FIG. 3, and that modifications to circuit may be possible in accordance with various embodiments.

In various embodiments, the buffering stage 350 and F2DP stage 355 may be located on the BTE ear pieces 345 themselves, in proximity to the one or more active electrodes. The adaptive amplifying stage 360 may, in some embodiments, be part of a sensing circuit located, for example, in an in-line controller housing. In other embodiments, the adaptive amplifying stage 360 may be located in the BTE ear piece with the buffering stage 350 and F2DP stage 355. Contact impedance with the skin 340 is represented schematically as resistor elements leading into the buffering stage, with biosignals represented as voltage source Vs, and noise as voltage source V_{CM-Noise} originating from the body 335.

To reduce the effect of motion artifact created by contact impedance fluctuation and cable sway, the buffering stage 350 may utilize an ultra-high input impedance buffer with unity gain. This effectively converts the high impedance signal lines to low impedance ones making it robust to the change of the capacitance on the wires when motion occurs. Conventionally, putting the buffer circuit directly on the electrodes is often done to minimize the inherent capacitance of the signal wires. This may not be desirable as there is limited space for the electrodes. In at least one embodiment, as long as the behind-the-ear sensing system 100 can keep the inherent capacitance small and stable, putting the circuit directly on the electrode is not needed. This is done by shielding the connection between each electrode and its buffer by using a micro-coax shielded cable.

In the second stage (F2DP stage 355), to ensure robustness against environmental interference, the weak and overlapped signals are pre-amplified before being driven to the cables to the sensing circuit. Conventional AE with positive gain usually face challenges with gain mismatch among electrodes because of the different of contact impedance. By dividing into Buffering 350 and F2DP 355 stages, gain mismatch is eliminated as input impedance of F2DP is effectively close to 0 Thus, contact impedance does not affect the gain in the next stages. Before preamplifying, the DC component in the signal has to be removed with a second-order Sallen-Key High Pass Filter so that only the AC signals are amplified.

Additionally, F2DP further increases Common-Mode Rejection Ratio (CMRR) over a conventional Driven Right Leg circuit (DRL) alone. The F2DP stage 355 employs a cross-connection topology where only one gain resistor is needed to set the gain for two feed-forward instrumentation amplifiers in our F2DP. After F2DP, fully differential and preamplified signals are produced making them robust against environment interference while driving the cables to the sensing circuit.

Stage 3 (adaptive amplifying stage 360) is configured to adaptively amplify signals with significant amplitude range differences, such as those between EEG/EOG and EMG signals. As previously described, the differences in amplitude may lead to signal saturation at the ADC on the sensing circuit when EMG signal is amplified with the same gain with EEG/EOG signal Thus, in various embodiments, the adaptive amplifying stage 360 may be configured to dynamically adjust gain in real-time of both EEG/EOG and EMG signals so that both small EEG/EOG and large EMG signal are captured with high resolution.

In various embodiments, the adaptive amplifying stage 360 may be put either in the ear pieces 345 or the sensing circuit, or both in at least one embodiment, the adaptive amplifying stage 360 may be implemented on the sensing circuit and use fixed gain amplifiers on the earpieces to pre-amplify the signal. This may reduce the number of wires nm from the ear pieces 345 to the in-line controller. In further embodiments, the adaptive amplifying stage 360 may further be implemented on the sensing circuit to ensure high quality signals. The behind-the-ear sensing system 100 may utilize a programmable gain instrumentation amplifier controlled by one or more AGC algorithms. AGC algorithms for the control of the adaptive amplifying stage 360 have been previously described.

In some embodiments, AGC may provide the ability to handle the large amplitude difference among different signals. For example, the AGC algorithm may change the amplifier's gain adaptively depending on the amplitude of the captured signal. The AGC provides at least two benefits: (1) the AGC is fast-responding, and (2) the AGC is lightweight. The fast response reduces the number of captured signals that will be lost during the transition of different gain levels. Thus, implementing the AGC on the firmware level will reduce the response time by cutting the communication delay for sending signals out. Additionally, AGC is light-weight so that it will not interfere with the main signal streaming.

FIGS. 4A-4C depict sample signals as they traverse through the different stages of the 3CA circuit described above.

FIG. 4A is a graph 400A depicting raw and filtered signals before and after the buffering stage of the 3CA circuit, in accordance with various embodiments. The graph 400A depicts a mixed signal, with a large-amplitude EMG event 405 reflected at the beginning of the signal. Eye blinks 410 are represented as peaks in the mixed signal, and motion artifacts are present in the highlighted window motion artifact duration 415. As previously described, the buffering stage may be configured to suppress the motion artifacts, for example, while the patient is walking or moving. Motion artifacts may be introduced by movement of the sensors as well as movement of the BTE device and cables. As depicted, it is shown that only the motion artifacts are suppressed with buffering (depicted in darker line), while the mixed biosignals are faithfully captured. In the un-buffered mixed signal, the eye blinks 410 may not be distinguishable from ones created by motions.

FIG. 4B is a graph 400B depicting a signal spectrum of the mixed signal before and after the F2DP stage of the 3CA circuit, in accordance with various embodiments. The graph 400B shows that electrical line noises (at 60 Hz) and its harmonics are further suppressed by 24 dB than using DRL alone. FIG. 4C is a graph 400C depicting the mixed signal with and without AGC at the adaptive amplifying stage of the 3CA circuit, in accordance with various embodiments. As shown in the top signal, amplifier saturation is frequency reached when a EMG events occur and gain is set based on the EEG/EOG signals. The bottom graph depicts the mixed signal when AGC is applied, and as shown, EMG events are able to be captured without saturation of the ADC. In the embodiments depicted, the gain is set according to an AGC algorithm in which a peak envelope detector (PED) window is small (W=10) while no EMG events occur such that the peak envelope detector can react quickly to increases amplitude, while a large window (W=100) may be utilized while an EMG event is occurring so as to avoid gain oscillations.

FIG. 5A is a graph 500A depicting oversampling and averaging of a saccadic eye movement (EOG) signal, in accordance with various embodiments. As previously described, and as shown in FIG. 5A, by utilizing OAA, similar SNR as a sampling rate of 1000 kHz may be maintained via OAA, while reducing total current draw of the sensing circuit. FIG. 5B illustrates comparative graphs 500B of EEG, vertical EOG, and horizontal EOG signals, in accordance with various embodiments. As previously described, FIG. 5B illustrates various EEG, vEOG, and hEOG signals as produced using different techniques. The top row of graphs depicts EEG, vEOG, and hEOG as obtained from a mixed signal after BPF for respective EEG, vEOG, and hEOG frequencies. The middle row depicts EEG, vEOG, and hEOG signals as obtained after using a combination of BPF and a transfer learning function, such as a convolutional autoencoder, as previously described. The bottom row depicts “ground-truth” EEG, vEOG, and hEOG signals, as captured by a PSG recording device with electrodes on the scalp of a patient.

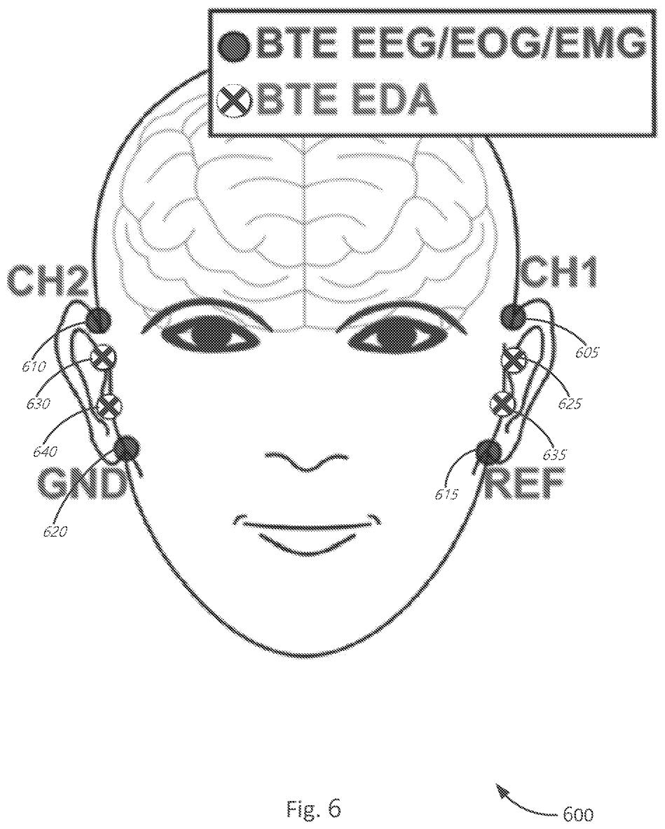

FIG. 6 is a schematic depiction of an example electrode arrangement 600 on a human head. In various embodiments, the electrode arrangement may include a first channel first electrode 605, second channel first electrode 610, a first channel second electrode 615, a second channel second electrode 620, a first channel third electrode 625, a second channel third electrode 630, a first channel fourth electrode 635, and second channel fourth electrode 645. It should be noted that the electrode arrangement 600 is schematically illustrated in FIG. 6, and that modifications to the electrode arrangement, including the number of electrodes, may be possible in accordance with various embodiments.

In various embodiments, biosignals may be captured BTE via a respective set of four electrodes for each ear. In some embodiments, the captured biosignals may include EEG, EOG, EMG, and EDA. Each respective set of four electrodes may include two different pairs of electrodes configured to capture a different set of subset of biosignals. In some embodiments, a first pair of electrodes may be used to capture EEG, EOG, and EMG signals from BTE A second pair of electrodes may be configured to capture EDA from BTE.

For the mixed signals, a first signal electrode (first channel first electrode 605) and reference electrode (first channel second electrode 615) may be placed at the upper and lower parts of the left ear piece, respectively. The second signal (second channel first electrode 610) and ground/bias electrode (second channel second electrode 620) of the driven right leg circuit may be placed at the upper and lower parts of the right earpiece, respectively. The respective pairs of electrodes are kept as far apart as possible to capture higher voltage changes With this arrangement, both electrodes on two ears can pick up EEG and EMG signal. Thus, to capture EEG, we place two electrodes, i.e. channel 1 (first channel) on the left ear and channel 2 (second channel) on the right ear, in the upper part of the area behind the ears so we could capture the EEG signal from the mid-brain area.

In some embodiments, for the eyes' signals (EOG), the signal electrode on the left ear may detect vEOG signals (i.e. eyes blinks and up/down eyes movements) while the electrode on the right ear may detect hEOG signals (i.e. left/right eyes movements). To captme EOG, the vertical and horizontal distance between each pair of electrodes, are maximized. Thus, the reference electrode 615 is placed in the lower part of behind the left ear area, close to the mastoid bone. With this setup, channel 1 can pick up eye blinks and up/down movements while channel 2 can capture eyes' left and right movements. In addition, both channel 1 and 2 can capture most of the facial muscle activity which links to the muscle group beneath the area behind the ears. As EEG, EOG and EMG are biopotential signals, two signal electrodes, a reference and a common ground, may be utilized to capture each of the biosignals.

For EDA, two pairs of sensing electrodes are utilized on both ears to capture signals generated on two sides on the body, a first channel third electrode 625 and first channel fourth electrode 635, and a second channel third electrode 630 and second channel fourth electrode 640. The BTE location works well to capture EDA as BTE has high sweat gland density Sweat gland activity, however, is not symmetric between two halves of the body. Thus, two electrodes are placed on each ear to reliably capture EDA.

FIG. 7 is a schematic block diagram of the control module of the BTE system 700, in accordance with various embodiments. The control module 710 may include MCU 715, communication chipset 720, and an integrated AFE 725. The control module 710 may be coupled to a left ear piece 705a and right ear piece 705b. It should be noted that the system 700 is schematically illustrated in FIG. 7, and that modifications to the system 700 may be possible in accordance with various embodiments.

In various embodiments, the control module 710 may include an in-line controller, as previously described. The control module 710 may, accordingly, be configured to be worn by the patient, along with ear pieces 705a, 705b In some embodiments, all or part of the control module 710 may be part of one or more of the ear pieces 705a, 705b, while in other embodiments, the control module 710 may be separate from the ear piece 705a, 705b assembly, and coupled to the ear pieces via cabling.

As previously described, the control module 710 may include on-board processing. In the depicted embodiments, the on-board processing may be an MCU 715 configured to execute pre-processing of the collected biosignals from the respective ear pieces 705a, 705b. The control module 710 may further comprise a communication chipset 720 configured to stream biosignal data to on-board storage and/or a host machine to which the control module 710 may be coupled via a wireless or wired connection. The communication chipset may include, without limitation, a Bluetooth chipset and/or Wi-Fi chipset. The control module 710 may further include an integrated AFE 725. As previously described, the integrated AFE 725 may include various circuitry for pre-processing of the biosignals, including various filters and amplifiers described above. In some embodiments, the integrate AFE 725 may include all or part of the 3CA circuitry, including OAA circuitry, F2DP circuitry, and adaptive amplification circuitry.

FIG. 8 is a schematic representation of a machine learning model 800 for the BTE system, in accordance with various embodiments. As previously described, the depicted model 800 shows each convolutional layer specified by a tuple of (kernel size, number of filters, stride size). The temporal and spectral feature separators are represented by the left and right tracks of the model respectively Temporal features are extracted using a low-parameter and small stride kernel that can identify longer, more simple trends and spectral features using a high-parameter, large stride kernel to learn more complex spectral features without learning overlapping trends. In some examples, each model may be trained using logcosh as the loss distance function, which is similar to mean squared error, but less sensitive to large differences in outliers, and the Adam optimizer with a learning rate of 0.01.

Models may be trained for 100 epochs on each signal type with shuffled 30 s epochs from all subjects, then used to predict the separated signal for each subject with each 30 s epoch in chronological order. Once both temporal and spectral separators have been trained, the weights from each are transferred to the combined model that adds the outputs of the two before comparison to the target signal. The combined learner uses the same loss and optimizer but with a reduced learning rate of 10E−6. Before use in the model, both the signals are normalized such that each 30 s epoch has a mean of 0 and a standard deviation of 1, signals are all aligned in time between the PSG and the wakefulness signal and the wakefulness signal is down sampled to 200 hz to match the PSG signal sampling rate. Thus, in various embodiments, the ML model 800 may be configured to classify a patient's wakefulness level based on features identified and extracted.

FIG. 9 is a table of features 900 that may be extracted from signals captured by the behind-the-ear sensing and stimulation system, in accordance with various embodiments. As previously described, features may be identified by an ML model and features selected and ranked according to relevance to a wakefulness classification.

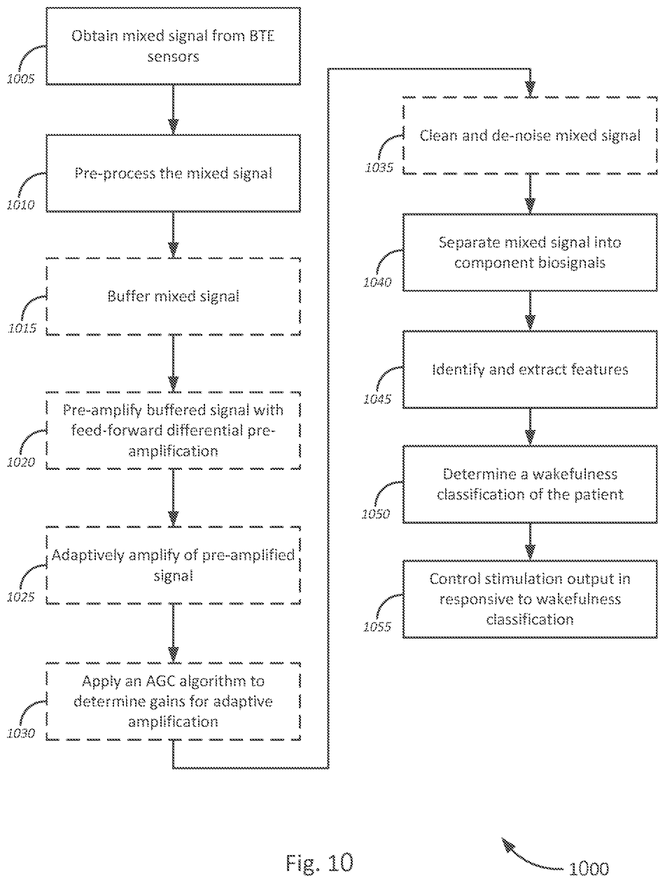

FIG. 10 is a method 1000 for wakefulness classification utilizing a BTE system, in accordance with various embodiments. The method 1000 begins, at block 1005, by obtaining a mixed biosignal from one or more sensors placed BTE of a patient. As previously described, BTE sensors may be provided for the detection of various biosignals, including EEG, EOG, EMG, and EDA. The one or more sensors may be placed behind the ear of the patient, and signals collected from the BTE location of the patient. The biosignals, as obtained directly from the sensors, may be a raw signal. As previously described, BTE sensors may be configured to be housed in a BTE earpiece, and coupled to the patient via the BTE earpieces, and positioned in an arrangement and spacing behind the ears of the patient as previously described.

The method 1000 continues, at block 1010, by pre-processing the mixed biosignal. In various embodiments, the raw mixed biosignal may be pre-processed via pre-processing circuitry in the ear piece, control module, such as an in-line controller, and/or in a sensing circuit. Pre-processing may include filtering, noise removal, and further conditioning of the signal for further downstream processing. In some embodiments, pre-processing may include 3CA as previously described, comprising a buffering stage, F2DP stage, and adaptive amplification stage. In yet further embodiments, pre-processing may include OAA of the mixed signal, as previously described.

Accordingly, at block 1015, the method 1000 continues by buffering the raw mixed biosignal. As previously described, in various embodiments, buffering may include passing the mixed signal through a buffering stage of a 3CA circuit. Buffering the signal may include utilizing an ultra-high input impedance buffer with unity gain. This effectively converts the high impedance signal lines to low impedance ones making it robust to the change of the capacitance on the wires when motion occurs. Thus, noise artifacts introduced by motion and cable sway may be removed.

At block 1025, the method continues by performing F2DP of the buffered mixed signal. As previously described, in various embodiments. In some embodiments, the F2DP stage of the 3CA circuit may be configured to perform pre-amplification of the buffered signal. In some embodiments, gain of the pre-amplification stage may be chosen so as to utilize the full dynamic range of an analog to digital converter (ADC) of the sensing circuit. Thus, F2DP may be configured to ensure robustness against environmental interference, the weak and overlapped signals are pre-amplified before being driven to the cables to the sensing circuit. Additionally, F2DP further increases Common-Mode Rejection Ratio (CMRR) over a conventional Driven Right Leg circuit (DRL) alone. F2DP employs a cross-connection topology where only one gain resistor is needed to set the gain for two feed-forward instrumentation amplifiers in our F2DP. After F2DP, fully differential and preamplified signals are produced making them robust against environment interference while driving the cables to the sensing circuit.

At block 1025, the method 1000 continues by adaptively amplifying the pre-amplified signal. The adaptive amplification stage may be configured to compensate for significant amplitude range differences between various biosignals, such as between EEG/EOG signals and EMG signals. As previously described, the differences in amplitude may lead to signal saturation at the ADC on the sensing circuit when EMG signal is amplified with the same gain with EEG/EOG signal. Thus, in various embodiments, the adaptive amplification may comprise dynamically adjusting gain in real-time of both EEG/EOG and EMG signals so that both small EEG/EOG and large EMG signal are captured with high resolution.

Thus, at block 1030, an AGC algorithm may be applied to determine gains for adaptive amplification. As previously described, in various embodiments, AGC algorithms may be applied to dynamically adjust the gain of the adaptive amplifier circuit according to an AGC algorithm. In some embodiments, the AGC algorithm may be configured to adjust gain according to EEG/EOG signals and EMG events. For example, when no significant EMG events are occurring, the EEG/EOG signals may be kept as maximum gain. The AGC 131 logic may further be configured to react quickly to abrupt increases in amplitude of EMG events (e.g., quickly attenuate an EMG signal), and react slowly to decreases in amplitude while an EMG event is ongoing to avoid gain oscillation.

At block 1035, the pre-processed mixed signal may be cleaned and de-noised. In various embodiments, as previously described, cleaning and de-noising of the mixed signal may include removal of motion artifacts and de-noising based on motion/positional signals and contact impedance signals, filtering, and other noise removal techniques.

At block 1040, the method 1000 may continue by separating the cleaned mixed signal into component biosignals. In various embodiments, as previously described, separating the biosignal may include BPF the mixed signal, using a combination BPF and a transfer learning function 170c of an ML model. Thus, by utilizing these techniques, mixed signal may be separated into EOG, EEG, EMG, and EDA biosignals.

At block 1045, the method continues by identifying and extracting features via a ML model. As previously described, an ML model may be configured to identify and extract various features of the component biosignals, such as, without limitation, EOG, EEG, EMG, and EDA. The ML model may further select and rank relevant features to wakefulness classification. At block 1050, the ML model may determine a wakefulness classification of the patient based on the features of the individual biosignals in some embodiments, the wakefulness determination may indicate a wakefulness level of the patient. Wakefulness level may be represented on a normalized scale configured to quantify a wakefulness of the patient. In one example, the wakefulness level may be normalized on a scale from 0 to 1, where 0 indicates a state of sleep or microsleep, and 1 indicates an awake state. In some embodiments, the scale may indicate an estimated probability that the patient is in a sleep and/or microsleep state, with 0 being 100% certainty, and 1 being 0% certainty. Alternatively, the ML model may be configured to determine whether the patient 180 is in an awake state, sleep state, and/or in a state of microsleep.

The method 1000 further includes, at block 1055, controlling a stimulation output responsive to the wakefulness classification. For example, as previously described, if it is determined by the ML model that the patient is in a state of microsleep, a stimulation output may be activated to provide stimulation to the patient Stimulation output may include, without limitation, an array of electrodes, bone conductance speakers, antennas, light emitters, and various other similar components. In some embodiments, RF and EM stimulation from the antennas, electrodes, and/or magnetic coils of the stimulation output may be directed to desired parts of the brain utilizing beamforming or phase array techniques. Additionally, acoustic and ultra-sound signals can be produced through a bone conductance speaker to transmit audio instruction to the patient, or to provide auditory stimulation to the patient, such as an alarm sound. In further embodiments, the light source may be an adjustable frequency light source, such as a variable frequency LED or other laser. Thus, the frequency of the light source may be changed. Moreover, the light source may be positioned to provide light stimulation to the eyes, or to expose skin around the head to different frequencies of light. In yet further embodiments, as previously described the stimulation output may be incorporated, at least in part, in a separate stimulation assembly. The stimulation assembly may include, for example, and without limitation, a mask, goggles, glasses, cap, hat, visor, helmet, or headband.

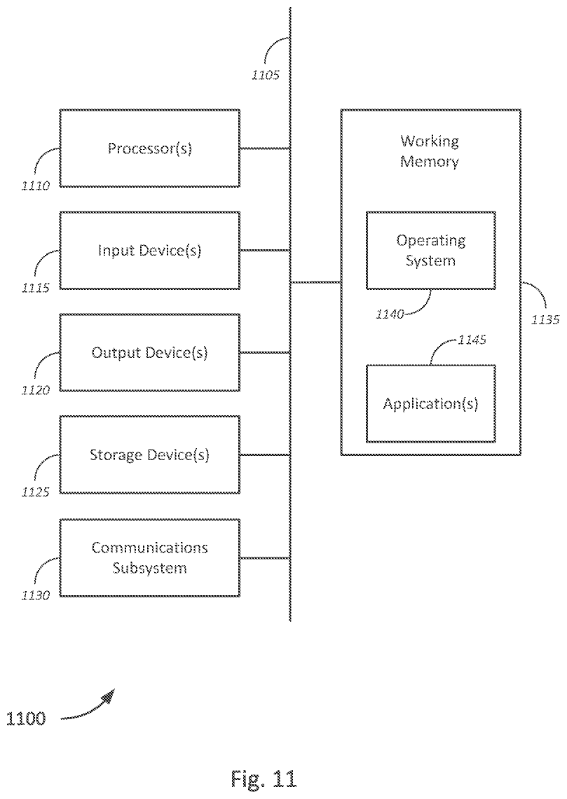

FIG. 11 is a schematic block diagram of a computer system for behind-the-ear sensing and stimulation, in accordance with various embodiments. The computer system 1100 is a schematic illustration of a computer system (physical and/or virtual), such as the control module, in-line controller, or host machine, which may perform the methods provided by various other embodiments, as described herein. It should be noted that FIG. 11 only provides a generalized illustration of various components, of which one or more of each may be utilized as appropriate. FIG. 11, therefore, broadly illustrates how individual system elements may be implemented in a relatively separated or relatively more integrated manner.

The computer system 1100 includes multiple hardware (or virtualized) elements that may be electrically coupled via a bus 1105 (or may otherwise be in communication, as appropriate). The hardware elements may include one or more processors 1110, including, without limitation, one or more general-purpose processors and/or one or more special-purpose processors (such as microprocessors, digital signal processing chips, graphics acceleration processors, and microcontrollers); one or more input devices 1115, which include, without limitation, a mouse, a keyboard, one or more sensors, and/or the like; and one or more output devices 1120, which can include, without limitation, a display device, and/or the like.

The computer system 1100 may further include (and/or be in communication with) one or more storage devices 1125, which can comprise, without limitation, local and/or network accessible storage, and/or can include, without limitation, a disk drive, a drive array, an optical storage device, solid-state storage device such as a random-access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer system 1100 may also include a communications subsystem 1130, which may include, without limitation, a modem, a network card (wireless or wired), an IR communication device, a wireless communication device and/or chipset (such as a Bluetooth™ device, an 802.11 device, a WiFi device, a WiMax device, a WWAN device, a low-power (LP) wireless device, a Z-Wave device, a ZigBee device, cellular communication facilities, etc.). The communications subsystem 1130 may permit data to be exchanged with a network (such as the network described below, to name one example), with other computer or hardware systems, between data centers or different cloud platforms, and/or with any other devices described herein. In many embodiments, the computer system 1100 further comprises a working memory 1135, which can include a RAM or ROM device, as described above.

The computer system 1100 also may comprise software elements, shown as being currently located within the working memory 1135, including an operating system 1140, device drivers, executable libraries, and/or other code, such as one or more application programs 1145, which may comprise computer programs provided by various embodiments, and/or may be designed to implement methods, and/or configure systems, provided by other embodiments, as described herein. Merely by way of example, one or more procedures described with respect to the method(s) discussed above may be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code may be encoded and/or stored on a non-transitory computer readable storage medium, such as the storage device(s) 1125 described above. In some cases, the storage medium may be incorporated within a computer system, such as the system 1100. In other embodiments, the storage medium may be separate from a computer system (i.e., a removable medium, such as a compact disc, etc.), and/or provided in an installation package, such that the storage medium can be used to program, configure, and/or adapt a general purpose computer with the instructions/code stored thereon. These instructions may take the form of executable code, which is executable by the computer system 1100 and/or may take the form of source and/or installable code, which, upon compilation and/or installation on the computer system 1100 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, single board computers, FPGAs, ASICs, and SoCs) may also be used, and/or particular elements may be implemented in hardware, software (including portable software, such as applets, etc.), or both. Further, connection to other computing devices such as network input/output devices may be employed.

As mentioned above, in one aspect, some embodiments may employ a computer or hardware system (such as the computer system 1100) to perform methods in accordance with various embodiments of the invention According to a set of embodiments, some or all of the procedures of such methods are performed by the computer system 1100 in response to processor 1110 executing one or more sequences of one or more instructions (which may be incorporated into the operating system 1140 and/or other code, such as an application program 1145 or firmware) contained in the working memory 1135. Such instructions may be read into the working memory 1135 from another computer readable medium, such as one or more of the storage device(s) 1125. Merely by way of example, execution of the sequences of instructions contained in the working memory 1135 may cause the processor(s) 1110 to perform one or more procedures of the methods described herein.

The terms “machine readable medium” and “computer readable medium,” as used herein, refer to any medium that participates in providing data that causes a machine to operate in a specific fashion. In an embodiment implemented using the computer system 1100, various computer readable media may be involved in providing instructions/code to processor(s) 1110 for execution and/or may be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 1125. Volatile media includes, without limitation, dynamic memory, such as the working memory 1135. In some alternative embodiments, a computer readable medium may take the form of transmission media, which includes, without limitation, coaxial cables, copper wire and fiber optics, including the wires that comprise the bus 1105, as well as the various components of the communication subsystem 1130 (and/or the media by which the communications subsystem 1130 provides communication with other devices). In an alternative set of embodiments, transmission media can also take the form of waves (including, without limitation, radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 1110 for execution. Merely by way of example, the instructions may initially be carried on a magnetic disk and/or optical disc of a remote computer A remote computer may load the instructions into its dynamic memory and send the instructions as signals over a transmission medium to be received and/or executed by the computer system 1100. These signals, which may be in the form of electromagnetic signals, acoustic signals, optical signals, and/or the like, are all examples of carrier waves on which instructions can be encoded, in accordance with various embodiments of the invention.

The communications subsystem 1130 (and/or components thereof) generally receives the signals, and the bus 1105 then may carry the signals (and/or the data, instructions, etc. carried by the signals) to the working memory 1135, from which the processor(s) 1110 retrieves and executes the instructions. The instructions received by the working memory 1135 may optionally be stored on a storage device 1125 either before or after execution by the processor(s) 1110.

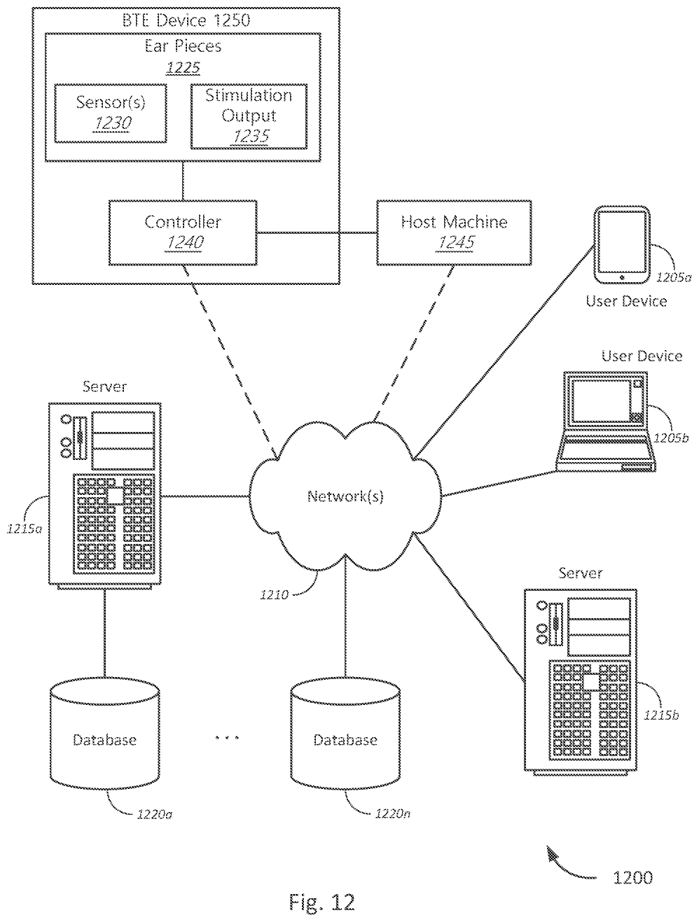

FIG. 12 is a schematic block diagram illustrating system 1200 of networked computer devices, in accordance with various embodiments. The system 1200 may include one or more user devices 1205. A user device 1205 may include, merely by way of example, desktop computers, single-board computers, tablet computers, laptop computers, handheld computers, edge devices, wearable devices, and the like, running an appropriate operating system. User devices 1205 may further include external devices, remote devices, servers, and/or workstation computers running any of a variety of operating systems. A user device 1205 may also have any of a variety of applications, including one or more applications configured to perform methods provided by various embodiments, as well as one or more office applications, database client and/or server applications, and/or web browser applications. Alternatively, a user device 1205 may include any other electronic device, such as a thin-client computer, Internet-enabled mobile telephone, and/or personal digital assistant, capable of communicating via a network (e.g., the network(s) 1210 described below) and/or of displaying and navigating web pages or other types of electronic documents. Although the exemplary system 1200 is shown with two user devices 1205a-1205b, any number of user devices 1205 may be supported.

Certain embodiments operate in a networked environment, which can include a network(s) 1210. The network(s) 1210 can be any type of network familiar to those skilled in the art that can support data communications, such as an access network, core network, or cloud network, and use any of a variety of commercially-available (and/or free or proprietary) protocols, including, without limitation, MQTT, CoAP, AMQP, STOMP, DDS, SCADA, XMPP, custom middleware agents, Modbus, BACnet, NCTIP, Bluetooth, Zigbee/Z-wave, TCP/IP, SNA™, IPX™, and the like. Merely by way of example, the network(s) 1210 can each include a local area network (“LAN”), including, without limitation, a fiber network, an Ethernet network, a Token-Ring™ network and/or the like; a wide-area network (“WAN”); a wireless wide area network (“WWAN”); a virtual network, such as a virtual private network (“VPN”); the Internet; an intranet: an extranet; a public switched telephone network (“PSTN”); an infra-red network, a wireless network, including, without limitation, a network operating under any of the IEEE 802.11 suite of protocols, the Bluetooth™ protocol known in the art, and/or any other wireless protocol; and/or any combination of these and/or other networks. In a particular embodiment, the network may include an access network of the service provider (e.g., an Internet service provider (“ISP”)). In another embodiment, the network may include a core network of the service provider, backbone network, cloud network, management network, and/or the Internet.

Embodiments can also include one or more server computers 1215. Each of the server computers 1215 may be configured with an operating system, including, without limitation, any of those discussed above, as well as any commercially (or freely) available server operating systems. Each of the servers 1215 may also be running one or more applications, which can be configured to provide services to one or more clients 1205 and/or other servers 1215.

Merely by way of example, one of the servers 1215 may be a data server, a web server, orchestration server, authentication server (e.g., TACACS, RADIUS, etc.), cloud computing device(s), or the like, as described above. The data server may include (or be in communication with) a web server, which can be used, merely by way of example, to process requests for web pages or other electronic documents from user computers 1205. The web server can also run a variety of server applications, including HTTP servers, FTP servers, CGI servers, database servers, Java servers, and the like. In some embodiments of the invention, the web server may be configured to serve web pages that can be operated within a web browser on one or more of the user computers 1205 to perform methods of the invention.

The server computers 1215, in some embodiments, may include one or more application servers, which can be configured with one or more applications, programs, web-based services, or other network resources accessible by a client. Merely by way of example, the server(s) 1215 can be one or more general purpose computers capable of executing programs or scripts in response to the user computers 1205 and/or other servers 1215, including, without limitation, web applications (which may, in some cases, be configured to perform methods provided by various embodiments). Merely by way of example, a web application can be implemented as one or more scripts or programs written in any suitable programming language, such as Java™, C, C#™ or C++, and/or any scripting language, such as Perl, Python, or TCL, as well as combinations of any programming and/or scripting languages. The application server(s) can also include database servers, including, without limitation, those commercially available from Oracle™, Microsoft™, Sybase™, IBM™, and the like, which can process requests from clients (including, depending on the configuration, dedicated database clients, API clients, web browsers, etc.) running on a user computer, user device, or customer device 1205 and/or another server 1215.

In accordance with further embodiments, one or more servers 1215 can function as a file server and/or can include one or more of the files (e.g., application code, data files, etc.) necessary to implement various disclosed methods, incorporated by an application running on a user computer 1205 and/or another server 1215. Alternatively, as those skilled in the art will appreciate, a file server can include all necessary files, allowing such an application to be invoked remotely by a user computer, user device, or customer device 1205 and/or server 1215.

It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 1220a-1220n (collectively, “databases 1220”). The location of each of the databases 1220 is discretionary: merely by way of example, a database 1220a may reside on a storage medium local to (and/or resident in) a server 1215a (or alternatively, user device 1205). Alternatively, a database 1220n can be remote so long as it can be in communication (e.g., via the network 1210) with one or more of these. In a particular set of embodiments, a database 1220 can reside in a storage-area network (“SAN”) familiar to those skilled in the art. In one set of embodiments, the database 1220 may be a relational database configured to host one or more data lakes collected from various data sources. The databases 1220 may include SQL, no-SQL, and/or hybrid databases, as known to those in the art. The database may be controlled and/or maintained by a database server.

The system 1200 may further include ear pieces 1225, one or more sensors 1230, stimulation output 1235, controller 1240, host machine 1245, and BTE device 1250. As previously described, with respect to other embodiments, the controller 1240 may include a communication subsystem configured to transmit a pre-processed mixed signal to the host machine 1245 for further processing. In some embodiments, the controller 1240 may be configured to transmit biosignals directly to the host machine 1245 via a wired or wireless connection, while in other embodiments the controller may be configured to transmit the biosignals to the host machine via the communications network 1210.

One will appreciate that the example of wakefulness monitoring with the behind-the-ear monitoring system is only a single example of potential uses system and is provided herein for the sake of example and explanation. Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to certain structural and/or functional components for ease of description, methods provided by various embodiments are not limited to any single structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in sequentially for ease of description, unless the context dictates otherwise, various procedures may be reordered, added, and/or omitted in accordance with various embodiments. Moreover, the procedures described with respect to one method or process may be incorporated within other described methods or processes, likewise, system components described according to a specific structural architecture and/or with respect to one system may be organized in alternative structural architectures and/or incorporated within other described systems. Hence, while various embodiments are described with—or without-certain features for ease of description and to illustrate exemplary aspects of those embodiments, the various components and/or features described herein with respect to one embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A system comprising:
a behind-the-ear wearable device comprising
an ear piece configured to be worn behind an ear of a patient;
one or more sensors coupled to the ear piece and configured to be in contact with the skin of the patient behind the ear of the patient;
a first processors coupled to the one or more sensors; and
a first computer readable medium in communication with the first processor, the first computer readable medium having encoded thereon a first set of instructions executable by the first processor to:
obtain, via the one or more sensors, a first signal, wherein the first signal comprises one or more combined biosignals, and
transmit the first signal;
a host machine coupled to the behind-the-ear wearable device, the host machine further comprising:
a second processor, and
a second computer readable medium in communication with the second processor, the second computer readable medium having encoded thereon a second set of instructions executable by the second processor to:
obtain, via the behind-the-ear wearable device, the first signal;
separate the first signal into one or more individual biosignals;
identify, via a machine learning model, one or more features associated with a wakefulness state,
extract the one or more features from each of the one or more individual biosignals; and
determine, based on the one or more features extracted from the one or more individual biosignals, a wakefulness classification of the patient.

2. The system of claim 1, wherein the one or more individual biosignals includes at least one of an electroencephalogram (EEG) signal, electrooculography (EOG) signal, electromyography (EMG) signal, and electrodermal activity (EDA) signal.

3. The system of claim 1 further comprising a stimulation output array, the stimulation output array comprising at least one of a light source, speaker, electrode, antenna, or magnetic coil.

4. The system of claim 3, wherein the ear piece further includes the stimulation output array.

5. The system of claim 3, wherein the second set of instructions is further executable by the second processor to:
control operation of the stimulation output array based on the wakefulness classification,
wherein if the wakefulness classification is indicative of a microsleep state, controlling the operation of the stimulation output array includes activating one or more of the at least one of the light source, speaker, electrode, antenna, or magnetic coil of the stimulation output array.

6. The system of claim 3, wherein the first set of instructions is further executable by the first processor to:
obtain, from the host machine, the wakefulness classification; and
control the operation of the stimulation output array based on the wakefulness classification, wherein if the wakefulness classification is indicative of a microsleep state, controlling the operation of the stimulation output array includes activating at least one of the light source, speaker, electrode, antenna, or magnetic coil of the stimulation output array.

7. The system of claim 1, further comprising a three-fold cascaded amplifying circuit, the three-fold cascaded amplifying circuit further comprising:
a buffering stage configured to remove motion artifacts from the first signal;
a feed forward differential pre-amplification stage configured to receive the first signal from the buffering stage, and pre-amplify the first signal utilizing a feed-forward topology to suppress electrical line noise, and
an adaptive amplification stage configured to receive the first signal from the feed forward differential pre-amplification stage, the adaptive amplification stage further configured to dynamically adjust a first amplifier gain based on the amplitude of at least one of the one or more combined biosignals.

8. The system of claim 7, wherein the first set of instructions is further executable by the first processor to:
set the first amplifier gain to a first gain level in response to determining that an amplitude of an electromyography signal of the one or more individual biosignals is below a threshold during a first time window;
set the first amplifier gain to a second gain level lower than the first gain level in response to determining that an amplitude of the electromyography signal of the one or more individual biosignals is above a threshold during the first time window, and for the duration of a second time window longer than the first window.

9. The system of claim 1, wherein the first set of instructions is further executable by the first processor to:
sample the first signal obtained from the one or more sensors at a first sampling rate to produce a first signal at the first sampling rate; and
average two or more samples of the first signal to produce a first signal at a second sampling rate than the first sampling rate.

10. The system of claim 1, wherein separating the first signal into one or more individual biosignals further comprises:
applying a respective bandpass filter for each of the one or more individual biosignals to the first signal, each respective bandpass filter further comprising a respective bandpass frequency associated with each of the one or more individual biosignals.

11. The system of claim 10, wherein separating the first signal into one or more individual biosignals further comprises:
recovering, via a transfer learning model built from a ground-truth signal, components of each of the one or more individual biosignals from frequency ranges that overlap with other individual biosignals of the one or more individual biosignals.

12. The system of claim 10, wherein determining the wakefulness classification of the patient further comprises determining whether the patient is in a microsleep state or an awake state.

13. The system of claim 10, wherein determining the wakefulness classification of the patient further comprises quantifying, via the machine learning model, a wakefulness level based on the captured biosignals from behind-the-ears, wherein the wakefulness level indicates an estimated probability that the patient is in a microsleep state.

14. An apparatus comprising:
a processor; and
a computer readable medium in communication with the processor, the computer readable medium having encoded thereon a set of instructions executable by the processor to:
obtain, via one or more behind-the-ear sensors, a first signal collected from behind the ear of a patient, the first signal comprising one or more combined biosignals,
separate the first signal into one or more individual component biosignals;
identify, via a machine learning model, one or more features associated with a wakefulness state for each of the one or more individual component biosignals;
extract the one or more features from each of the one or more individual biosignals, and
determine, based on the one or more features extracted from the one or more individual biosignals, a wakefulness classification of the patient.

15. The apparatus of claim 14, wherein the one or more individual biosignals includes at least one of an electroencephalogram (EEG) signal, electrooculography (EOG) signal, electromyography (EMG) signal, and electrodermal activity (EDA) signal.

16. The apparatus of claim 14, wherein the set of instructions is further executable by the processor to:
control operation of a stimulation array based on the wakefulness classification, wherein the stimulation array includes at least one of the light source, speaker, electrode, antenna, or magnetic coil of the stimulation output array,
wherein if the wakefulness classification is indicative of a microsleep state, controlling the operation of the stimulation array includes activating one or more of the at least one of the light source, speaker, electrode, antenna, or magnetic coil of the stimulation output array.

17. The apparatus of claim 14, wherein separating the first signal into one or more individual biosignals further comprises:
applying a respective bandpass filter for each of the one or more individual biosignals to the first signal, each respective bandpass filter further comprising a respective bandpass frequency associated with each of the one or more individual biosignals.

18. The apparatus of claim 14, wherein separating the first signal into one or more individual biosignals further comprises:
recovering, via a transfer learning model built from a ground-truth signal, components of each of the one or more individual biosignals from frequency ranges that overlap with other individual biosignals of the one or more individual biosignals.

19. A method comprising:
obtaining, via one or more behind-the-ear sensors, a first signal from behind the ears of a patient, the first signal comprising one or more combined biosignals;
separating, via a machine learning model, the first signal into one or more individual component biosignals;
identifying, via the machine learning model, one or more features associated with a wakefulness state for each of the one or more individual component biosignals;
extracting the one or more features from each of the one or more individual biosignals; and
determining, via the machine learning model, a wakefulness classification of the patient based on the one or more features extracted from the one or more individual biosignals.

20. The method of claim 19 further comprising:
controlling operation of a stimulation array based on the wakefulness classification, wherein the stimulation array includes at least one of the light source, speaker, electrode, antenna, or magnetic coil of the stimulation output array,
wherein if the wakefulness classification is indicative of a microsleep state, controlling the operation of the stimulation array includes activating one or more of the at least one of the light source, speaker, electrode, antenna, or magnetic coil of the stimulation output array.
